SEQUENCE LISTING (53 sequences)

SEQ ID NO: 1 - DNA, 28 bp, Artificial Sequence
  SYNTHETIC DNA PRIMER
aaggtaccggagagttcaacacggatgg

SEQ ID NO: 2 - DNA, 28 bp, Artificial Sequence
  SYNTHETIC DNA PRIMER
ttaagcttcctgcccttgctctgcctgg

SEQ ID NO: 3 - DNA, 19 bp, Artificial Sequence
  SYNTHETIC DNA PRIMER
ggaaacagctatgaccatg

SEQ ID NO: 4 - DNA, 20 bp, Artificial Sequence
  SYNTHETIC DNA PRIMER
gagcatatgggattgaaagg

SEQ ID NO: 5 - DNA, 20 bp, Artificial Sequence
  SYNTHETIC DNA PRIMER
aaattaaccctcactaaagg

SEQ ID NO: 6 - DNA, 20 bp, Artificial Sequence
  SYNTHETIC DNA PRIMER
accaacacacagtgtcatgg

SEQ ID NO: 7 - DNA, 20 bp, Artificial Sequence
  SYNTHETIC DNA PRIMER
cagaatcaagctcaacgtgg

SEQ ID NO: 8 - DNA, 20 bp, Artificial Sequence
  SYNTHETIC DNA PRIMER
tccctgtcgaaggactctgg

SEQ ID NO: 9 - DNA, 20 bp, Artificial Sequence
  SYNTHETIC DNA PRIMER
ccaaccagaggccccaccat

SEQ ID NO: 10 - DNA, 20 bp, Artificial Sequence
  SYNTHETIC DNA PRIMER
ggctaaccttgttcttcagg

SEQ ID NO: 11 - DNA, 20 bp, Artificial Sequence
  DNA PRIMER
aaacgccatcaagtacctgg

SEQ ID NO: 12 - DNA, 21 bp, Artificial Sequence
  SYNTHETIC DNA PRIMER
tcggtgcagaatagtctcgtt

SEQ ID NO: 13 - DNA, 20 bp, Artificial Sequence
  SYNTHETIC DNA PRIMER
ccctgatttgtgcaatgagg

SEQ ID NO: 14 - DNA, 20 bp, Artificial Sequence
  SYNTHETIC DNA PRIMER
tgggttcatcctcgcagagg

SEQ ID NO: 15 - DNA, 20 bp, Artificial Sequence
  SYNTHETIC DNA PRIMER
tcatgccgacccgcagtccc

SEQ ID NO: 16 - DNA, 20 bp, Artificial Sequence
  SYNTHETIC DNA PRIMER
gttaagagatcatctccacc

SEQ ID NO: 17 - DNA, 29 bp, Artificial Sequence
  SYNTHETIC DNA PRIMER
ggtaccctcttcttgcatctgtctgtgac

SEQ ID NO: 18 - DNA, 28 bp, Artificial Sequence
  SYNTHETIC DNA PRIMER
agatctgtgactctggatgatcagtggc

SEQ ID NO: 19 - DNA, 20 bp, Artificial Sequence
  SYNTHETIC DNA PRIMER
gcccaggagtcaggctaacc

SEQ ID NO: 20 - DNA, 20 bp, Artificial Sequence
  SYNTHETIC DNA PRIMER
acacactcagatgtctcacc

SEQ ID NO: 21 - DNA, 20 bp, Artificial Sequence
  SYNTHETIC DNA PRIMER
ttggggtcttcacaggtgcc

SEQ ID NO: 22 - DNA, 20 bp, Artificial Sequence
  SYNTHETIC DNA PRIMER
gggttcccagctcgccagcc

SEQ ID NO: 23 - DNA, 20 bp, Artificial Sequence
  SYNTHETIC DNA PRIMER
ccgcgagagccgtctctagg

SEQ ID NO: 24 - DNA, 20 bp, Artificial Sequence
  SYNTHETIC DNA PRIMER
agcctcgggcacatgaaagg

SEQ ID NO: 25 - DNA, 20 bp, Artificial Sequence
  SYNTHETIC DNA PRIMER
gaacccgagaaaggcaaagg

SEQ ID NO: 26 - DNA, 20 bp, Artificial Sequence
  SYNTHETIC DNA PRIMER
tgttaactgtacctttgcgg

SEQ ID NO: 27 - DNA, 21 bp, Artificial Sequence
  DNA PRIMER
ccacaggaagcagctgcatgg

SEQ ID NO: 28 - DNA, 29 bp, Artificial Sequence
  SYNTHETIC DNA PRIMER
aattaattaagctcgcgataagaggagcc

SEQ ID NO: 29 - DNA, 42 bp, Artificial Sequence
  SYNTHETIC DNA PRIMER
aattaattaaggcacacagacctggtgcagggcggtgagtcc

SEQ ID NO: 30 - DNA, 28 bp, Artificial Sequence
  SYNTHETIC DNA PRIMER
ttaattaaatgcagatggcggacgcgaa

SEQ ID NO: 31 - DNA, 28 bp, Artificial Sequence
  SYNTHETIC DNA PRIMER
ttaattaattttattaggaaaggacagt

SEQ ID NO: 32 - DNA, 20 bp, Artificial Sequence
  SYNTHETIC DNA PRIMER
ccctttgtagcactggttgg

SEQ ID NO: 33 - DNA, 20 bp, Artificial Sequence
  SYNTHETIC DNA PRIMER
caattgttccaggaaccagg

SEQ ID NO: 34 - DNA, 20 bp, Artificial Sequence
  SYNTHETIC DNA PRIMER
gtcaagactataagtacagg

SEQ ID NO: 35 - DNA, 20 bp, Artificial Sequence
  SYNTHETIC DNA PRIMER
gcggttccatcttccagcgg

SEQ ID NO: 36 - DNA, 26 bp, Artificial Sequence
  SYNTHETIC DNA PRIMER
gatatctgatgagaggagctacctgc

SEQ ID NO: 37 - DNA, 20 bp, Artificial Sequence
  SYNTHETIC DNA PRIMER
gtcctcttcaattttgcagc

SEQ ID NO: 38 - DNA, 20 bp, Artificial Sequence
  SYNTHETIC DNA PRIMER
gggttgggaggagtaggagg

SEQ ID NO: 39 - DNA, 20 bp, Artificial Sequence
  SYNTHETIC DNA PRIMER
ggttttgaagataggagcgg

SEQ ID NO: 40 - DNA, 20 bp, Artificial Sequence
  SYNTHETIC DNA PRIMER
ctagacactattccagatgg

SEQ ID NO: 41 - DNA, 20 bp, Artificial Sequence
  SYNTHETIC DNA PRIMER
cactatccctccttggctgg

SEQ ID NO: 42 - DNA, 20 bp, Artificial Sequence
  SYNTHETIC DNA PRIMER
tcattctagaatcagactgg

SEQ ID NO: 43 - DNA, 20 bp, Artificial Sequence
  SYNTHETIC DNA PRIMER
ctagttggagagaagagcgg

SEQ ID NO: 44 - DNA, 1966 bp, Artificial Sequence
  SYNTHETIC LUCIFERASE CONSTRUCTKpnI-PacI-AscI-LUCIFERASE-PvuII CASSETTE
ggtaccttaattaaggcgcgccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataagcagctg

SEQ ID NO: 45 - DNA, 6 bp, Artificial Sequence
  KPN1 RESTRICTION ENDONUCLEASE SITE
ggtacc

SEQ ID NO: 46 - DNA, 8 bp, Artificial Sequence
  PAC1 RESTRICTION ENDONUCLEASE SITE
ttaattaa

SEQ ID NO: 47 - DNA, 8 bp, Artificial Sequence
  ASC1 RESTRICTION ENDONUCLEASE SITE
ggcgcgcc

SEQ ID NO: 48 - DNA, 6 bp, Artificial Sequence
  PVUII RESTRICTION ENDONUCLEASE SITE
cagctg

SEQ ID NO: 49 - DNA, 966 bp, Artificial Sequence
  SYNTHETIC FRT-TET-FRT CASSETTE FRT-TETOCMVEX1-IN1(PARTIAL)-FRT
cgatcggaagttcctatactttctagagaataggaacttctcgggtgatctgactgatcccgcagattggagatcgccgcccgtgcctgccgattgggtgcagatctcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatctacagctcttgatggaaaccttctgcagagaacaatggcccctctgggttattttaaacttcactctaagccttctccacctgctctgctgtctctaattgctccagaaacagcgtcattcacaactggattattaatgaactgagcggggagcctcgggcacatgaaaggggacttcagacttggggaagtcttcgaaggacccaaccagaggtgggttttgttgttttaagacaaggtctcatgtggcacagacaggctggcctcaaatccataagtatgcctgagagctggatttacatgtgtttgttaccacacctggtactggcacacatgcagggtactgaagttcctatactttctagagaataggaacttcggcgcgcc

SEQ ID NO: 50 - DNA, 820 bp, Artificial Sequence
  SYNTHETIC FRT-TET-FRT CASSETTE FRT-TETOCMVEX1-(PARTIAL)-FRT
cgatcggaagttcctatactttctagagaataggaacttctcgggtgatctgactgatcccgcagattggagatcgccgcccgtgcctgccgattgggtgcagatctcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatctacagctcttgatggaaaccttctgcagagaacaatggcccctctgggttattttaaacttcactctaagccttctccacctgctctgctgtctctaattgctccagaaacagcgtcattcacaactggattattaatgaactgagcggggagcctcgggcacatgaaaggggacttcagacttggggaagttaattaagaagttcctatactttctagagaataggaacttcggcgcgcc

SEQ ID NO: 51 - DNA, 6 bp, Artificial Sequence
  RESTRICTION ENDONUCLEASE SITE
cgatcg

SEQ ID NO: 52 - DNA, 1781 bp, Artificial Sequence
  SYNTHETIC DEST CASSETTE WITH+ORIENTATION
ggcgcgcccaagctggctaggtaagcttgatcaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctccgttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgatctagagggcccgcggttcgaaggtaagcctaggcgcgcc

SEQ ID NO: 53 - DNA, 1781 bp, Artificial Sequence
  SYNTHETIC DEST CASSETTE WITH - ORIENTATION
ggcgcgcctaggcttaccttcgaaccgcgggccctctagatcaaccactttgtacaagaaagctgaacgagaaacgtaaaatgatataaatatcaatatattaaattagattttgcataaaaaacagactacataatactgtaaaacacaacatatccagtcactatggtcgacctgcagactggctgtgtataacggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatccagatctttacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgccggatcctaactcaaaatccacacattatacgagccggaagcataaagtgtaaagcctggggtgcctaatgcggccgccatagtgactggatatgttgtgttttacagtattatgtagtctgttttttatgcaaaatctaatttaatatattgatatttatatcattttacgtttctcgttcagcttttttgtacaaacttgttgatcaagcttacctagccagcttgggcgcgcc

(19) United States
(12) Patent Application Publication
(10) Pub. No.: US 2013/0318645 A1
(43) Pub. Date: Nov. 28, 2013

(54) Methods and Vectors for Gene Targeting With Inducible Specific Expression

(71) Applicant: Georgia Regent University, (US)

(72) Inventor: Christophe Poirier, Westfield, IN (US)

(21) Appl. No.: 13/900,065

(22) Filed: May 22, 2013

Related U.S. Application Data
(60) Provisional application No. 61/650,291, filed on May 22, 2012.

(51) Int. Cl. C12N 15/90; C12Q 1/68; C12N 15/85

(52) U.S. Cl. CPC C12N 15/907; C12N 15/85; C12Q 1/6897
USPC 800/21; 435/320.1; 435/6.18; 435/325; 435/354; 435/363

(57) ABSTRACT

A method, called GETWISE, for targeting mouse genes is described. GETWISE is designed to increase the frequency of homologous recombination, facilitate screening, widen the applicability of engineered animals and circumvent intrinsic gene targeting problems. GETWISE utilizes the principle of modulating gene expression by targeting tetracycline-responsive elements into a specific locus. In GETWISE alleles, control of gene expression is transferred from the endogenous to a tetracycline-inducible promoter. Endogenous promoters now control expression of the reporter gene luciferase. Breeding of GETWISE carriers with tTA/rtTA carriers enables investigators to modulate gene expression in a ubiquitous or tissue-specific manner, depending on the presence of doxycycline. GETWISE enables the study of loss or gain of gene expression in any tissue of choice within a single mouse strain. GETWISE enables the analysis of the gene expression pattern with the luciferase assay.

### CROSS REFERENCE TO RELATED APPLICATIONS

This application claims benefit of and priority to U.S. Ser. No. 61/650,291 filed on May 22, 2012, which is incorporated by reference in its entirety.

### REFERENCE TO SEQUENCE LISTING

The Sequence Listing submitted May 22, 2013 as a text file named “GRU_{—}2011_{—}016_ST25.txt,” created on May 16, 2013, and having a size of 19,990 bytes is hereby incorporated by reference pursuant to 37 C.F.R. §1.52(e)(5).

### STATEMENT REGARDING FEDERALLY SPONSORED RESEARCH OR DEVELOPMENT

This invention was made with government support under Grant Numbers HL080675, HL083327 and HL067307 awarded by the National Institutes of Health. The U.S. Government has certain rights in the invention.

### FIELD OF THE INVENTION

The present invention relates to the field of gene targeting techniques and in particular to a technique of gene targeting with inducible specific expression.

### BACKGROUND OF THE INVENTION

The development of genetically engineered animals is an arduous and multi-staged process, associated with certain difficulties. Because loss-of-function and gain-of-function mutations in some genes are known to result in unrelated disorders (Grino et al., J Clin Endocrinol Metab, 66:754-761 (1988); Kim, et al., J Med Genet, 42:666-672 (2005); La Spada, et al., Nature, 352:77-79 (1991); Muenke, et al., Nat Genet, 8:269-274 (1994)), investigators studying the role of a gene have to use animals both deficient and overexpressing the gene. Problems such as early embryonic lethality due to function deficiency (Gu, et al., Science 265:103-106 (1994)) or lethality due to the toxicity of ectopic expression (Brunkow, et al., Genes Dev, 5:1092-1101 (1991)) can force the scientist to develop time-specific models for the loss or gain of gene expression. Tissue specific manipulation of gene expression is needed to elucidate the role of a gene in certain diseases. Generally, different models require generation of different lines of genetically engineered animals. Thus, there is a need in the art for a cost-effective and time-effective strategy, allowing the generation of animal models of total loss/gain of expression, conditional loss/gain of expression and tissue-specific loss/gain of expression, using only one line of genetically engineered animals.

It is an object of the invention to provide compositions and methods for the manipulation of gene expression.

### SUMMARY OF THE INVENTION

Compositions and methods for the targeting of genes with insertion vectors for inducible, tissue specific expression are described. The components and methods of this system are referred to as Gene Targeting With Inducible Specific Expression (GETWISE) (FIGS. 1-2).

In some embodiments the disclosed system includes methods for targeting genes to allow multiple possibilities for activation or inactivation of expression, specifically in a tissue of choice and/or a time of choice. Targeting is achieved by transferring the control of gene expression to a conditional promoter and by simultaneous transfer of a reporter gene under the control of an endogenous promoter. Combination of these two features provides versatility in application and speed in screening.

In certain embodiments, the disclosed system reassigns control over transcription of the targeted gene to the tetracycline-inducible promoter (TIP), which results in a ubiquitous loss of gene expression in transgenic GETWISE animals. These GETWISE animals enable the investigator to study the effect of gene knockout and to monitor the pattern of gene expression. For the ubiquitous or tissue-specific modulation of gene expression, investigators can set crosses between GETWISE animals and tTA/rtTA-expressing animals. The choice of tissue selected for specific gene expression does not have to be made before the GETWISE animal is generated. Because modulation of the gene expression in the offspring is triggered by exposure to doxycycline, the disclosed system has a built-in ability to circumvent problems such as early embryonic lethality due to gene deficiency, or due to ectopic over-expression. Prior knowledge of gene deficit or gene over-expression effects is not required and does not affect the way GETWISE animals are engineered.

The scientist studying the function of a mutant protein can benefit from developing transgenic mice with the GETWISE system because GETWISE mice no longer express the wild-type protein. Traditionally, the study of the mutant protein function required breeding between the mutant carriers and knockouts to abolish the expression of wild-type protein. The versatility and efficiency of the GETWISE system makes it an attractive alternative to the currently used techniques for gene manipulation.

One embodiment provides GETWISE insertion vectors designed for gene-specific activation or inactivation of expression which facilitate fast and effective cloning and identification of homologous, recombinant genes.

Another embodiment provides a method for the application of GETWISE insertion vectors in the development of Embryonic Stem (ES) cell clones necessary for the generation of transgenic animals which exhibit specific activation or inactivation of expression of the targeted gene.

A further embodiment provides a method for the application of GETWISE insertion vectors in the development of transgenic animals for the tissue-specific, inducible activation or inactivation expression of the targeted gene.

### BRIEF DESCRIPTION OF THE DRAWINGS

FIG. 1 is a schematic diagram showing three possible genotypes of transgenic mice offered by GETWISE.

FIGS. 2A-2C show a schematic diagram illustrating the design of a GETWISE targeting vector. FIG. 2A is a schematic illustrating the hypothetical targeted gene. A promoter is shown with an arrow on the left, exons are shown as open boxes. The targeting area should not exceed 6 kb so it can be easily amplified by PCR. It should not contain the promoter of the gene. The 3′ end of the targeting area should be located within an exon.

FIGS. 3A-3D show a schematic diagram illustrating the targeting of the Ppp1r16b gene (Timap). FIG. 3A is a schematic diagram illustrating the Timap gene. The Timap promoter is indicated by an arrow on the left, and 12 non-coding and coding exons are indicated by black and white boxes, respectively. FIG. 3B is a schematic diagram illustrating the targeted region of the gene, between exons 2 and 3. 2 ApaI sites are indicated by vertical arrows. FIG. 3C is a schematic diagram illustrating the targeting vector. The TIP on the left, indicated by a dotted arrow, contains tetracycline responsive elements. The entire Timap exon #1 is shown, indicated by a solid box, and a part of the Timap intron #1 is shown, indicated by a solid line. The single ApaI site within the homologous region is indicated by a vertical arrow. The 5′utr fragment of exon #3 is indicated by a black box. The luciferase reporter gene indicated by a grey box. loxP sites flanking the Pgk::NEO cassette are indicated by triangles. The Bluescript plasmid is shown as a circle, labeled KS. Thick arrows on FIGS. 3B and 3C indicate primers used to screen for homologous recombination. FIG. 3D is a schematic diagram illustrating the resulting GETWISE allele (TimapGETWISE). The primers described in FIGS. 4A-4D are shown as black, grey, and open triangles.

FIGS. 4A-4E illustrate the validation of GETWISE technique for expression of the Ppp1r16b (Timap) gene. FIG. 4A shows images of two agarose gels to demonstrate expression of luciferase (upper panel) from Timap promoter (Timap^{LUC}) and Capn1 (lower panel) in wild type (wt) and GETWISE embryoid bodies, respectively. RT-PCR was performed using primers marked with black triangles on FIG. 3D. FIG. 4B shows images of two agarose gels to demonstrate expression of Timap (upper panel) and Capn1 (lower panel) from TIP in embryoid bodies infected with GFP- or tTA-expressing adenoviruses, respectively. Results of doxycycline (DOX+) or vehicle control (−) are shown for each expression test. RT-PCR was performed using primers marked with grey triangles on FIG. 3D. The forward primer has been selected to avoid amplification of mRNA from Timap wild-type allele; it has been designed across the exon #1 and exon #3 junctions, thus by-passing exon #2. FIG. 4C is a histogram bar graph showing the analysis of Timap promoter-driven luciferase expression in +/GETWISE mice and Timap mRNA expression in wild-type mice, in brain and lung, respectively and the results of a Real-Time PCR assay, performed with primers from indicated as open triangles in FIG. 3D. Hprt was used as a control. Data are expressed as mean±sem. FIG. 4D shows images of two agarose gels to demonstrate the expression of Timap in mouse lung. The upper panel shows expression of Timap in wild-type (wt) mice, GETWISE/GETWISE mice, or GETWISE/GETWISE mice carrying Tek::rtTA transgene (rtTA transgene) and subjected to doxycycline (DOX) or vehicle control treatment. Expression was tested by RT-PCR, using primers marked with open triangles on FIGS. 3D, 4A, 4B, and 4D. The lower panel shows the expression of control Hprt genes. DNA ladders are on the left, with an arrow at 500 by DNA fragment. FIG. 4E shows the level of Timap induction in mouse lung. Heterozygous (+/GETWISE) mice carrying Tek::rtTA transgene (rtTA) were stimulated with doxycycline (+DOX) or vehicle control (−DOX). The level of Timap expression was measured by Real-Time PCR and expressed as mean±sem, the t-test p value is indicated.

FIGS. 5A-5D show a schematic diagram illustrating the targeting of the targeting of the Smpd3 gene. FIG. 5A is a schematic diagram of the Smpd3 gene. The Smpd3 promoter is indicated with a solid arrow, on the left. 9 exons are depicted as black (non-coding) and white (coding) boxes, respectively. FIG. 5B is a schematic diagram of the Smpd3 targeted region. PshAI and KpnI restriction sites used to linearize the targeting vector and for screening by inverse-PCR, respectively, are indicated by vertical arrows. FIG. 5C is a schematic diagram of the targeting vector. The TIP, containing tetracycline responsive elements, is indicated by a dotted arrow on the left. The entire Timap exon #1 is indicated by a solid box, and a part of Timap intron #1 is indicated by a solid line. KpnI sites used for screening by inverse-PCR are shown by vertical arrows. A single PshAI site within the homologous region is indicated by a vertical arrow. The 5′utr fragment of exon #3 is indicated by a black box. The luciferase reporter gene is indicated by a grey box. loxP sites flanking the Pgk::NEO cassette are indicated by triangles. The Bluescript plasmid is shown as a circle, labeled KS. Thick arrows on FIGS. 5B and 5C indicate primers used to screen for homologous recombination by inverse-PCR. FIG. 5D is a schematic diagram of the resulting GETWISE allele (Smpd3GETWISE).) The positions of primers used for the analysis in FIGS. 5E and 5F are indicated with black, and open triangles, respectively. FIG. 5E shows images of two agarose gels to demonstrate the expression of luciferase from the Smpd3 promoter (Smpd3^{LUC}, upper panel), tested by RT-PCR using primers marked with black triangles, in wild type (wt) and GETWISE (Getwise) embryoid bodies, respectively. The lower panel shows the expression of control Hprt genes. DNA ladders are on the left, with an arrow at 500 by DNA fragment. FIG. 5F shows images of two agarose gels to demonstrate the expression of Smpd3 from TIP (Smpd3^{TIP}, upper panel), detected by RT-PCR, using primers marked with open triangles, in embryoid bodies infected with GFP or tTA expressing adenoviruses and subjected to doxycycline (DOX+) or vehicle control (−), respectively. The lower panel shows the expression of control Hprt genes. DNA ladders are on the left, with an arrow at 500 by DNA fragment.

FIG. 6 shows a schematic diagram illustrating the generation of the pGETWISE1.1 and pGETWISE2.1 plasmids. From top to bottom, 4 different pGETWISE plasmids are shown. FRT sites are shown as diamonds marked with F. In pGETWISE1.1, TIP consists of 540 bp containing the tetracycline responsive element, indicated by a dotted arrow, from pRevTRE, 212 by of the entire Timap exon #1, and 132 by of the 5′ end sequence from Timap intron #1. The junction between exon#1 and intron#1 provides a complete splice donor site. In pGETWISE2.1, TIP consists of 540 by containing the tetracycline responsive element from pRevTRE, 190 by of the 5′end of Timap exon #1 followed by PacI restriction site, indicated by vertical arrows. The GATEWAY cassette, indicated by a dotted box, from pEF-DEST 51 has been cloned using an AscI restriction site, indicated by vertical arrows. (+) and (−) indicate the orientation of the cassette. The luciferase coding sequence followed by the SV40 late polyadenylation signal is indicated by a grey box. The neomycin resistance cassette is indicated by an open box. loxP restriction sites are indicated by open triangles. DNA cassettes used to build these plasmids are described in FIGS. 7-8.

FIGS. 7A-E show a schematic diagram illustrating the targeting of the Ppp1r12a (Mypt1) gene. FIG. 7A is a schematic diagram illustrating the Mypt1 gene. The position of Mypt1 promoter is shown with the solid arrow on the left. 26 exons of Mypt1 are depicted as black (non-coding) and white (coding) boxes, respectively. The first coding exon is exon #1. FIG. 7B is a schematic diagram illustrating the targeted region, including 2 AflII restriction sites, indicated by vertical arrows, used for gap creation. FIG. 7C is a schematic diagram illustrating 2 variants of the targeting vector. The bluescript sequence has been omitted due to the lack of space. The upper schematic shows cDNA coding for the constitutively active form of MYPT1, indicated by a striped box. The lower schematic shows the coding sequence from exon #1 with a complete splice donor, indicated by an open box. Both targeting vectors are designed for cloning into pGETWISE2.1(−) at a Pad restriction site. In both variants, vertical arrows show the AflII ligation site. A homologous region comprising ˜5.7 kb of the Mypt1 gene is inserted into and gapped with the AflII ligation site. Here and on FIG. 7B, thick horizontal arrows indicate the location of primers used to screen for homologous recombination by PCR. FIGS. 7D-E are schematic diagrams illustrating the genomic structures of the classic GETWISE allele (FIG. 7D, Mypt1 GETWISE) and mutant allele (FIG. 7E, Mypt1C/A), respectively. The Mypt1 promoter is indicated by a solid arrow. The Mypt1 promoter drives the expression of the luciferase reporter gene. TIP controls the expression of either wild-type MYPT1 isoforms (FIG. 7D) or constitutively active MYPT1 C/A mutant (FIG. 7E).

FIGS. 8A-D show a schematic diagram illustrating the targeting of the Sgms1 gene. FIG. 8A is a schematic diagram illustrating the Sgms1 gene. The positions of 2 major promoters for Sgms1 (P1 and P2) are shown with solid arrows. The exons are depicted as black (non-coding) and white (coding) boxes, respectively. Messengers transcribed from P1 and P2 have specific and common sequences originated from different exons, as indicated; specific exons are shown above or below the horizontal line for P1 and P2, respectively; common exons are shown 30 across the horizontal line). FIG. 8B is a schematic diagram illustrating the targeted region, with 2 AflII sites (used for gap creation) marked with vertical arrows. FIG. 8C is a schematic diagram illustrating the targeting vector. ˜4.9 kb of the Sgms1 homologous region was inserted into pGETWISE1.1(−) and gapped (vertical arrow shows the ligation site). Here, and on FIG. 7B, horizontal arrows indicate the position of primers used to screen for homologous recombination by PCR. FIG. 8D. GETWISE allele (Sgms1GETWISE) is shown. Sgms1 promoters (solid arrows) drive the expression of the luciferase reporter gene. TIP controls the expression of all SMS1 isoforms.

FIGS. 9A-C illustrate the targeting of Ncrna00085 (Bachelor). FIG. 9A is a schematic diagram illustrating the position of Bachelor promoter, indicated by the solid arrow on the left. 8 exons are depicted as black (non-coding) and white (coding) boxes. The asterisk (*) marks the end of exon #6 where alternative splicing occurs; addition of 2 nucleotides from intron #6 in mRNA increases the size of the coding sequence. 2 NdeI sites used for gap creation are marked with vertical arrows. FIG. 9B is a schematic diagram illustrating the targeting vector. ˜3.1 kb of the Bachelor homologous region was inserted into pGETWISE2.1(−) and gapped (vertical arrow indicates the site of ligation). Here and on FIG. 9A, thick horizontal arrows indicate the location of luciferase primers used to screen for homologous recombination by PCR. FIG. 9C. GETWISE allele (BachGETWISE) is shown. Bachelor promoter (solid arrow) drives the expression of the luciferase reporter gene. TIP controls the expression of all BACHELOR isoforms.

FIG. 10 is a schematic diagram illustrating multiplex screening. The scheme of the 96-well plate with the values of luciferase activity is shown. Only 1 clone, C9, rendered a value exceeding the average more than 25 fold. The result of the PCR screening for the DNA pooled from 8 rows (right) and 12 columns (bottom) are shown for Mypt1, Bachelor, and Sgms1. For Mypt1, 4 rows and 4 columns were positive. For Bachelor, 4 rows and 5 columns were positive. For Sgms1, 1 row and 1 column are positive. Clones at the intersections of positive rows and columns were thawed and expanded to confirm the genotype (16 clones for Mypt1, 20 clones for Bachelor, 1 clone for Sgms1).

FIGS. 11A-B show a schematic diagram illustrating the genotyping of homozygous GETWISE/GETWISE mice by linkage association. The parental ES cell line, originated from the mouse 129 strain, was used to generate GETWISE chimeric animals. Male chimeric mice were bred with females from the C57BL/6N (B6) strain; the agouti offspring were heterozygous B6/129. In +/GETWISE mice, wildtype (+) and GETWISE alleles have been inherited from the B6 strain and 129 strain, respectively. FIG. 11A is a schematic diagram illustrating that cross between +/GETWISE parents renders 3 genotypes: +/+, +/GETWISE, and GETWISE/GETWISE. To distinguish between +/GETWISE and GETWISE/GETWISE animals, one can select DNA markers M1 and M2. These markers need to fulfill 3 following conditions: 1) they have to be polymorphic between B6 and 129 strains; 2) they have to flank the targeted gene; 3) they have to be as close as possible to reduce the possibility of double crossing-over during meiosis. FIG. 11B is a pictorial diagram of agarose gels used for genotyping of M1 (left panel) and M2 (right panel) mice heterozygous and homozygous for B6 or 129 alleles, respectively. Mice homozygous for B6 alleles at M1 and M2 are homozygous for the wildtype allele (+/+) at the targeted gene. Mice heterozygous at M1 and M2 are heterozygous for GETWISE allele (+/GETWISE) at the targeted gene. Mice homozygous for 129 alleles at M1 and M2 are homozygous for the GETWISE allele (GETWISE/GETWISE) at the targeted gene.

FIGS. 12A-D illustrate strategies to genotype the TimapGETWISE allele. FIG. 12A is a schematic diagram of the Timap KO (Timap^{KO}) and Timap GETWISE (Timap^{GETWISE})alleles. In the KO allele exon #4 has been replaced by a neo cassette (white box). Exons #3 and #4 are separated by a ˜44 kb intron, making the 2 alleles tightly linked. FIG. 12B is an image of an agarose gel showing PCR genotyping for the KO allele, performed as described in (Heinzel et al., Eur J Immunol, 37:2562-2571, (2007)), with 3 primers located within intron #3, intron #4, and the NEO cassette, respectively. With these primers, it is possible to amplify a 342 by fragment with exon #4 and a 290 by fragment without exon #4 (KO allele). From left to right, 3 mice were genotyped; they were a) homozygous for the 342 by fragment (homozygous GETWISE); b) heterozygous; c) homozygous for the 290 by fragment (homozygous KO). DNA ladder is on the right with arrows at 400, 300, and 200 by DNA fragments, respectively. FIG. 12C is a bar graph histogram showing the relative amounts of mice homozygous for GETWISE mice heterozygous for GETWISE, as determined by Real-Time PCR. Copy number of the transgene was estimated in mice homozygous (black bar) or heterozygous (white bar) for GETWISE. Data are expressed as mean±sem; the t-test p value is indicated. As expected, mice homozygous for GETWISE carry twice more copies of the transgene than mice heterozygous for GETWISE. FIG. 12D is a bar graph histogram showing the relative amounts of mice homozygous for GETWISE and mice heterozygous for GETWISE, as determined by Luciferase expression in the lungs of heterozygous and homozygous GETWISE mice, respectively. Luciferase activity was measured for heterozygous (white bar) and homozygous (black bar) mice. Background luciferase activity was measured in wildtype mice. Data are expressed as mean ±sem; t-test analysis did not confirm the difference in luciferase activity as statistically significant.

FIGS. 13A-C show a schematic diagram illustrating the genotyping of homozygous GETWISE/GETWISE using sub-alleles. FIG. 13A is a schematic diagram illustrating the original GETWISE allele. The allele contains the NEO cassette, indicated by a box located between 2 loxP sites, indicated by triangles. The allele is referred to as NEO+. The locations of the primers (A, B, C) used for genotyping are indicated by horizontal arrows. Forward primer A is located at the 3′ end of luciferase. Forward primer B is located at the 3′ end of the NEO cassette. Reverse primer C is located within the KS vector. FIG. 13B is a schematic diagram illustrating how the generation of GETWISE mice without the NEO cassette is possible after breeding GETWISE mice with mice expressing CRE-recombinase in the germline (DNA recombination occurs between 2 loxP sites). The NEO cassette is deleted and a single loxP site remains, thus creating a sub-allele referred to as NEO−. FIG. 13C is a pictorial diagram of agarose gels used to distinguish NEO+ from NEO− sub-alleles in the offspring of GETWISE parents carrying the + and not carrying the − NEO cassette, by PCR. Mice heterozygous for GETWISE carry either one of NEO+ or NEO− sub-alleles; mice homozygous for GETWISE carry both NEO+ and NEO− sub-alleles.

FIG. 14 shows a schematic diagram illustrating a breeding scheme allowing re-expression of the target gene, using CRE transgenic mice.

### DETAILED DESCRIPTION OF THE INVENTION

I. Definitions

As used herein, the term “vector” refers to a molecule incorporating nucleic acid sequences encoding regulatory elements for transcription, translation, transcript stability, replication, and other functions as are known in the art. A vector may be a nucleic acid such as a plasmid or other DNA vector. The vector may comprise one or more genes in a linear or circularized configuration. The vector may also have a “plasmid backbone” that is involved in the production, manufacture, or analysis of a gene product. An “expression vector” is a vector that allows for production of a product encoded for by a nucleic acid sequence contained in the vector. For example, expression of a particular growth factor protein encoded by a particular gene. A “gene product” means products encoded by the nucleic acid sequences of the vector.

The term “coding region” refers to a portion of nucleic acid containing codons which may be translated into amino acids, although “stop codons” (TAG, TGA, or TAA) are not translated into an amino acids, but may also be considered to be part of a coding region. Unless stated otherwise, promoters, ribosome binding sites, transcriptional terminators, introns, and the like, are not considered part of a coding region. Coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions. In addition, a vector, polynucleotide, or nucleic acid embodiments may encode heterologous coding regions, either fused or unfused to a nucleic acid encoding a different heterologous polypeptide. Heterologous coding regions include without limitation specialized elements or motifs, such as a secretory signal peptide or a heterologous functional domains.

The term “regulatory element” refers to a DNA sequence that controls and regulates the transcription of another DNA sequence.

The term “promoter” refers to a nucleic acid sequence sufficient to direct transcription of a gene. Also included in the invention are those promoter elements which are sufficient to render promoter dependent gene expression controllable for cell type specific, tissue specific or inducible by external signals or agents. Promoters that are induced and cause a gene to be expressed following exposure or treatment of the cell with an agent, biological molecule, chemical, ligand, light, or the like that induces the promoter are commonly referred to as “inducible promoters” or “regulatable” promoters.

The term “nucleic acid” refers to both RNA and DNA including: cDNA, genomic DNA, plasmid DNA, condensed nucleic acid, or nucleic acid formulated with compounds able to prolong the localized bioavailability of a nucleic acid.

The term “stem cell” defines a cell with the ability to divide for indefinite periods in culture and give rise to specialized cells. Stem cells include, for example, somatic (adult) and embryonic stem cells. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell derived from the embryo that has the potential to become a wide variety of specialized cell types. An embryonic stem cell is one that has been cultured under in vitro conditions that allow proliferation without differentiation. Stem cells may be induced pluripotent stem cells. Pluripotent embryonic stem cells can be distinguished from other types of cells by the use of markers including, but not limited to, Oct-4, alkaline phosphatase, CD30, TDGF-1, GCTM-2, Genesis, Germ cell nuclear factor, SSEA1, SSEA3, and SSEA4. Stem cells may be of human or non-human origin. Non-limiting examples of non-human stem cells include non-human primate stem cells and mouse stem cells.

The term “transfection” refers to the introduction of a nucleic acid sequence into the interior of a membrane enclosed space of a living cell, including introduction of the nucleic acid sequence into the cytosol of a cell as well as the interior space of a mitochondria, nucleus or chloroplast. The nucleic acid may be in the form of naked DNA or RNA, associated with various proteins or the nucleic acid may be incorporated into a vector.

The term “gene targeting” refers to homologous recombination of DNA sequences between an exogenous DNA sequence and the chromosome of a host organism or host cell.

The term “target gene” refers the gene or DNA segment or nucleic acid molecule of interest that is to be targeted for inducible specific expression.

The term “host” refers to the cell or organism in which gene targeting occurs.

The term “reporter gene” refers to a gene that confers characteristics on the organisms expressing it that can be easily identified and measured. Reporter genes can induce visually identifiable characteristics, such as the expression of fluorescent or luminescent proteins. Examples include the gene that encodes jellyfish green fluorescent protein (GFP), which causes cells that express it to glow green under blue light, the enzyme luciferase (LUC), which catalyzes a reaction with luciferin to produce light, and the red fluorescent protein from the gene dsRed.

The term “selection marker” refers to a gene introduced into a cell, especially a bacterium or to cells in culture, which confers a trait suitable for artificial selection. Selection markers may be genes that can be expressed to convey an identifying factor or phenotype that is a readily observable and a distinguishable trait, usually an antibiotic or chemical resistance gene, that is able to be selected for based upon the marker gene's effect, i.e., resistance to an phenotype that makes the organism resistant or susceptible to a specific set of conditions. Selectable markers may be resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest.

The term “restriction site” refers to a recognition site or sites for a restriction endonuclease enzyme that catalyzes a double strand break in DNA at specific locations on a polynucleotide.

The term “homologous region” refers to a nucleotide sequence within the vector which is sufficiently similar to that of the targeted gene to facilitate homologous recombination, but which may be different from the targeted gene in certain ways.

The term “homologous recombination” refers to the process whereby an exogenous nucleic acid sequence with homology to a target sequence within a host undergoes recombination between the exogenous nucleic acid and the target nucleic acid, causing the delivered nucleic acid to be integrated into the host, thereby forming a homologously-recombined host.

The term “GETWISE transgenic mice” refers to transgenic mice that have been generated from homologous recombination with GETWISE inducible specific expression vectors. GETWISE transgenic mice typically exhibit a ubiquitous lack of expression of the target gene and constitutive expression of the reporter gene.

The term “exogenous” refers to a nucleic acid sequence that was introduced into a cell or organelle from an external source. Typically the introduced exogenous sequence is a recombinant sequence.

The term “exogenous promoter” refers to a nucleic acid sequence which controls and regulates the transcription of another DNA sequence, which was introduced into a cell or organelle from an external source.

The term “immediately contiguous” refers to a spatial location that is directly juxtapositional to another, such that direct contact between two adjacent entities may occur.

II. Vectors for Gene Targeting with Inducible Specific Expression

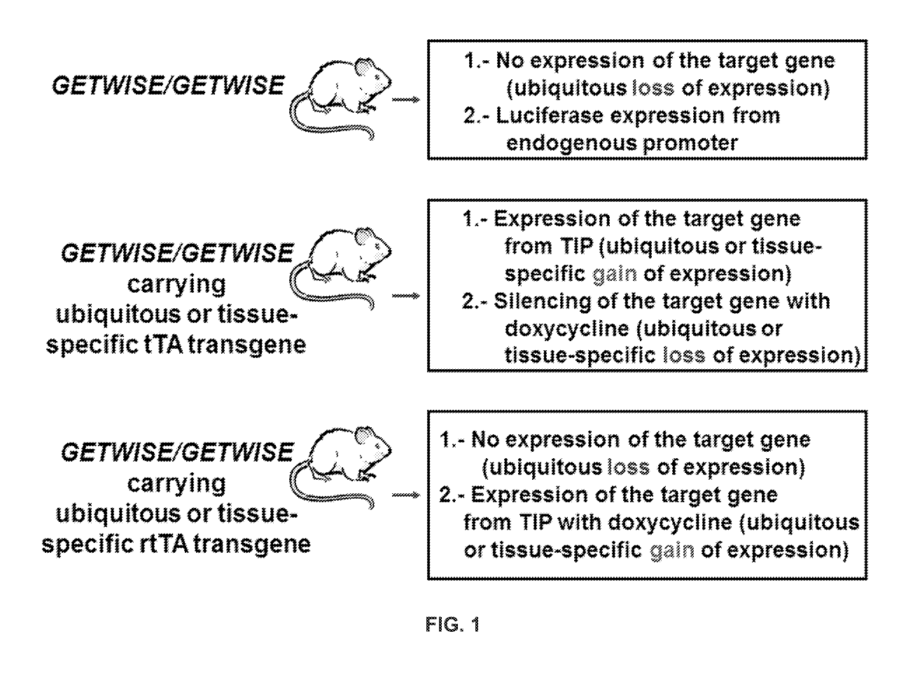

The GETWISE system provides a cost-effective and time-effective strategy allowing any type of tissue-specific and conditional manipulation of gene expression with only one line of genetically engineered animals (FIG. 1).

GETWISE constructs include a vector which can be linearized and inserted within a host cell genome. The vector typically includes a homologous region with a 5′ end and a 3′ end, the 3′ end of which is homologous to an exon within a target genomic gene, at least one endonuclease restriction site within positioned to promote in-frame homologous recombination of the vector with the target genomic gene, an exogenous and inducible promoter upstream of the homologous region, a reporter gene immediately contiguous with the 3′end of the homologous region and a selection marker gene downstream of the reporter gene. The vector may be designed to contain exons from the target gene between the inducible promoter and the homologous region, upstream of where the vector recombines with the target genomic gene.

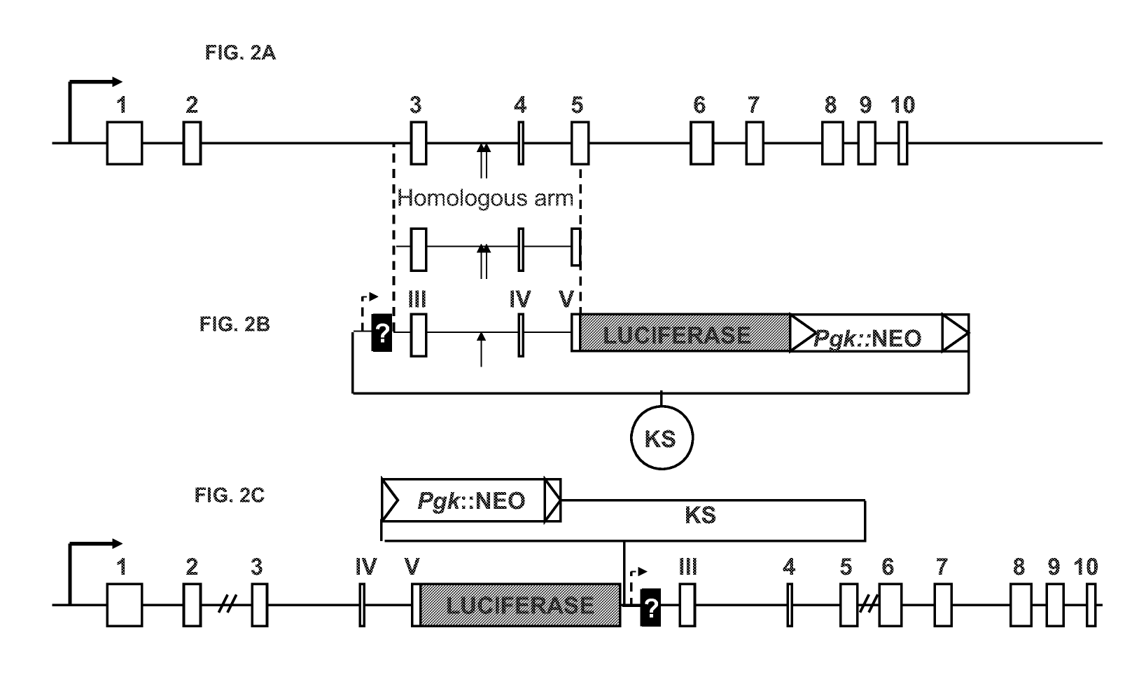

Homologous recombination of the GETWISE constructs can be initiated at the linearization site (FIGS. 2A-2C). Upon recombination, the entire GETWISE insertion vector can be inserted into the target gene, causing duplication of the homologous sequence. Following homologous recombination with the genomic target gene, expression of the reporter gene is controlled by a promoter of the genomic gene and expression of the target gene is controlled by the inducible promoter. Insertion vectors for the homologous recombination of target genes have been described previously (Hasty, et al., Mol Cell Biol, 11:4509-4517 (1991)). In one embodiment, insertion of the GETWISE vector is capable of generating null alleles, although the targeted gene is not deleted. Methods for the generation of null alleles by insertion vectors are well known in the art (Agoulnik, et al., Hum Mol Genet, 11:3047-3053 (2002); Chambrey, et al., Am J Physiol Renal Physiol, 289:F1281-1290 (2005); Dietrich, et al., PLoS One, 5:e9960 (2010); Folgueras, et al., Blood, 112:2539-2545 (2008); Gorlov, et al., Hum Mol Genet, 11:2309-2318 (2002); Guyon, et al., J Neurosci, 29:2528-2533 (2009); Ijuin, et al., Mol Cell Biol, 28:5184-5195 (2008); Kamat, et al, Endocrinology, 145:4712-4720 (2004); O'Neal, et al., Hum Mol Genet, 2:1561-1569 (1993); Rahuel, et al., Am J Physiol Renal Physiol, 294:F393-406 (2008); Rohozinski, et al., Genesis 32:1-7 (2002); Rudolph, et al., Transgenic Res, 2:345-355 (1993); Sales, et al., PLoS One, 6:e23261 (2011); Yan, et al., Mol Cell Biol 23:6798-6808 (2003)).

The elements of the GETWISE vectors are described in more detail below.

A. Inducible Promoters

The disclosed GETWISE vectors can transfer control of the targeted gene transcription to an exogenous, inducible promoter. In one embodiment the inducible promoter is a tetracycline-inducible promoter (TIP), whereby the TIP cassette contains the tetracycline responsive element (Gossen, et al., Science 268:1766-1769 (1995)), to allow the doxycycline-dependent manipulation of gene expression. Doxycycline is a member of the tetracycline antibiotics group.

When the inducible promoter is a tetracycline-inducible promoter (TIP), introduction of ubiquitous or tissue-specific tetracycline-controlled transactivators (tTA) to the animal background enables investigator to over-express the gene from embryonic stage onward in the absence of doxycycline (ubiquitous or tissue-specific gain of expression) and silence it upon the exposure to doxycycline (conditional loss of expression).

Furthermore, when the inducible promoter is a tetracycline-inducible promoter (TIP), introduction of the reverse tetracycline-controlled transactivators (rtTA) to the animal background enables the investigator to gain gene expression in the tissue of choice upon an exposure of the offspring to doxycycline (conditional gain of expression).

The principle of exploiting tetracycline-inducible system for gene conditional expression or silencing was described more than a decade ago (Gross, et al., Nature, 416:396-400 (2002); Shin, et al., Nature, 402:496-501 (1999); Tanaka, et al., Biol Psychiatry, 67:770-773 (2010)); however, replacement vectors were used and the strategy did not allow the targeting of genes with multiple promoters. Today, a number of mice carrying tissue-specific tTA, and rtTA transgenes are commercially available.

Commercially available transgenic mice expressing the rtTA or tTA transactivators can be obtained at www.findmice.org. The same ID number is assigned to different transgenic lines generated with the same promoter. Currently, 31 tissue-specific transgenic lines are available for rtTA expression, and 27 for tTA expression. A transactivator is also commercially available for complementary expression of opposite direction.

In one embodiment, GETWISE transgenes contain the tetracycline-inducible promoter (TIP), whereby TIP-driven transcription occurs from the original genomic sequence, allowing the expression of all splice variants.

In another embodiment, cDNA encoding a mutant protein can be put under the control of TIP.

B. Homologous Region

In preferred embodiments, the composition of GETWISE vectors is determined by the major structural features of the target gene. For example, GETWISE vectors are designed differently, depending on the presence of the coding sequence upstream of the homologous region (FIG. 2A-2C). The inducible promoter (e.g., TIP) is typically placed in front of the whole coding sequence after homologous recombination.

The sequence of the homologous region typically includes a region where two sites for the same restriction enzyme are positioned with no less than 100 base pairs (bp) between them. In certain embodiments, the internal sequence between these restriction sites can be excised to create a gap-vector. This gap can be repaired upon homologous recombination (Rudolph, et al., Transgenic Res, 2:345-355 (1993)), which allows screening for homologous recombination by PCR with primers located within the backbone of the vector and in the gap.

In other embodiments, the internal sequence between these restriction sites is not excised to create a gap-vector (no-gap vectors). Screening of no-gap vectors can be achieved either by Southern blot or inverse-PCR.

In a preferred embodiment, the targeted area of the target gene does not exceed 6 kilo base pairs (kb) and can be easily amplified by PCR. In the preferred embodiment, the targeted area of the target gene does not contain the promoter of the gene and the 3′ end of the targeting area is not located within an exon.

In certain embodiments, the GETWISE vector can be designed to accommodate the generation of vectors suitable to target genes with various different structures. In certain embodiments the targeted gene has multiple promoters.

C. Reporter Genes

The disclosed GETWISE vectors were designed to place a reporter gene under the control of the targeted gene promoter in host cells, to allow monitoring of target gene expression in the host cells. Useful reporter genes include alkaline phosphatase, green fluorescent protein (GFP), enhanced green fluorescent protein, destabilized green fluorescent protein, luciferase and beta-galactosidase.

In one embodiment the reporter gene is the firefly luciferase reporter gene. The luciferase enzyme reporter system offers a high range of linearity for quantitative measurements, and allows cell-specific detection with anti-luciferase antibodies by immunohistochemistry. Aside from the variety of options offered for gene manipulation, GETWISE mutants that carry the luciferase gene under the control of the target gene promoters also enable rapid, cost effective analysis of the target gene expression pattern.

The use of the reporter gene luciferase in GETWISE can provide new opportunities for the future analysis of engineered animals. In one embodiment, breeding GETWISE transgenic mice containing the luciferase reporter gene with mice other transgenic mice, expressing different conventional genetic markers, such as lacZ or GFP, under the control of certain gene promoters can enable monitoring of the effects of mutations resulting from GETWISE on these genes' expression without the concern of cross-interference of markers.

D. Selectable Marker

The disclosed GETWISE vectors can include selection markers to facilitate the selection of recombinant clones. Positive selection is provided when the marker conveys an advantage to the organism or cell possessing it, compared to those lacking it. Negative selection is provided when the marker conveys a relative disadvantage to an organism or cell possessing the marker.

In certain embodiments, positive selection can be provided, for example, by a gene conferring resistance to an antibiotic or other toxin so that in the presence of the toxin, cells lacking the resistance are less viable than cells possessing the resistance.

In other embodiments, negative selection is provided by a gene conferring sensitivity to a specific compound, so that cells possessing the gene are selectively killed in the presence of the toxin.

Positive selection may be achieved using resistance to antibiotics, such as kanamycin penicillin, streptomycin, ampicillin, zeomycin, or neomycin.

Antibiotic resistance genes that may be used as selectable markers are neomycin phosphotransferase II (nptII) and hygromycin phosphotransferase (hpt), gentamycin acetyltransferase (accC3) and bleomycin and phleomycin genes. In certain embodiments, the selection marker gene is flanked by loxP sites.

E. Vector Backbone

The disclosed GETWISE vectors can be based upon a plasmid vector. In certain embodiments the vector may be a commercially available plasmid vector. In one embodiment the vector backbone is the pBluescript or pBluescript II phagemid. The pBluescript vector backbone contains a polylinker sequence that is located within a LacZ controlled gene, designed to provide a blue color when expressed in bacteria. Because the inserts disrupt the coding region of the lacZ gene fragment, pBluescript phagemids containing inserts produce white colonies in the same bacterial strain. pBluescript vector backbones (+) and (−) are available with two polylinker orientations, designated as either KS or SK, respectively. In the KS orientation, the Kpn I restriction site is nearest the lacZ promoter and the Sac I restriction site is farthest from the lacZ promoter. In the SK orientation, the Sac I site is the closest restriction site to the lacZ promoter and the Kpn I site is the farthest.

Other examples of vector backbones include the pUC18 plasmid.

F. Exemplary GETWISE Vectors

Methods of engineering pre-made vectors for GETWISE gene-targeting are also disclosed. Specifically, the construction of GETWISE targeting vectors that involve the cloning of only 1 or 2 DNA fragments, depending upon the structure of the gene, is described (FIG. 2A-2C). Particularly described are the generation of 6 GETWISE alleles for 5 target genes, the development of one GETWISE mouse line, and validation of GETWISE efficiency and applicability.

The last generation vector for conditional knockouts, “Knockout-first” (Testa, et al., Genesis, 38:151-158 (2004)), the investigator needs to clone 3 DNA fragments; thus, the disclosed methods can diminish the number of molecular steps required for construction of the targeting vector.

The homologous region of the disclosed vectors can be created by PCR amplification of the target gene area (FIG. 2B). In a preferred embodiment 2 restriction sites within the homologous region enable the removal of the internal fragment to yield a single restriction site which is used to linearize the plasmid. The nature of the following sequence upstream of the inducible promoter (e.g. TIP) is dependent upon the presence of the coding sequence upstream of homologous region. If there is no coding sequence upstream, the sequence should contain only a splice donor. In one embodiment, there is a coding sequence upstream of homologous region, which is included as a genomic or cDNA fragment followed by a splice donor. In another embodiment, there is not a coding sequence upstream of the homologous region, and the last exon of the homologous region is be put in frame with the coding sequence of the reporter gene (e.g. luciferase) (FIG. 2C).

A chimeric mRNA, which is transcribed from the promoter of the targeted gene following homologous recombination is disclosed. This mRNA contains the complete sequences from exons upstream of, and including the homologous region, fused to the reporter (e.g. luciferase) coding sequence. From the inducible promoter (e.g. TIP), a conditional transcription of mRNA containing the entire coding sequence of the targeted gene is expected.

To enable efficient targeting of genes with different genomic structures, ready-to-use GETWISE vectors are disclosed. Specific embodiments are presented as proof of the ease and straightforwardness of the technique herein.

For genes with no coding sequence in the exons upstream the homologous region, pGETWISE1.1 can be used. For genes with coding sequence upstream the homologous region, pGETWISE2.1 can be used. For these genes, the luciferase sequence has to be in frame with the coding sequence upstream from the homologous region. The promoter of the targeted gene subsequently drives the expression of the luciferase fused to a fragment of the targeted protein.

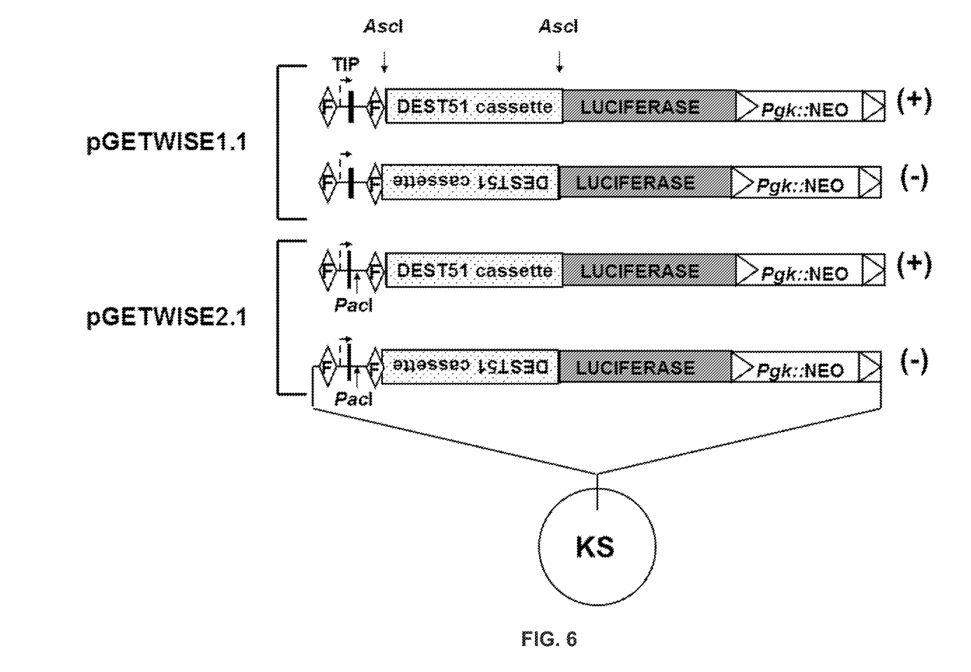

In pGETWISE2.1, the coding sequence from the upstream exons can be cloned into PacI site because TIP needs to be placed in front of the whole coding sequence after homologous recombination (FIGS. 2A-2C and 6). Regardless of the presence of coding sequence in homologous region or upstream, pGETWISE2.1 should be used when the investigator wants to re-express a mutant allele from the TIP. In this case, the mutant allele should be cloned at the PacI site.

The described methods were validated by generating mice carrying GETWISE mutation for Ppp1r16b/Timap, breeding with endothelial-specific rtTA carrier, and inducing Ppp1r16b/Timap with doxycycline. Ready-to-use GETWISE vectors were designed for targeting genes of different structures. It is demonstrated herein the generation of 5 additional GETWISE mutations in ES cells (Ppp1r12a/Mypt1, Ppp1r12a/Mypt1 constitutively active, Smpd3, Sgms1, and Ncrna00085/Bachelor).

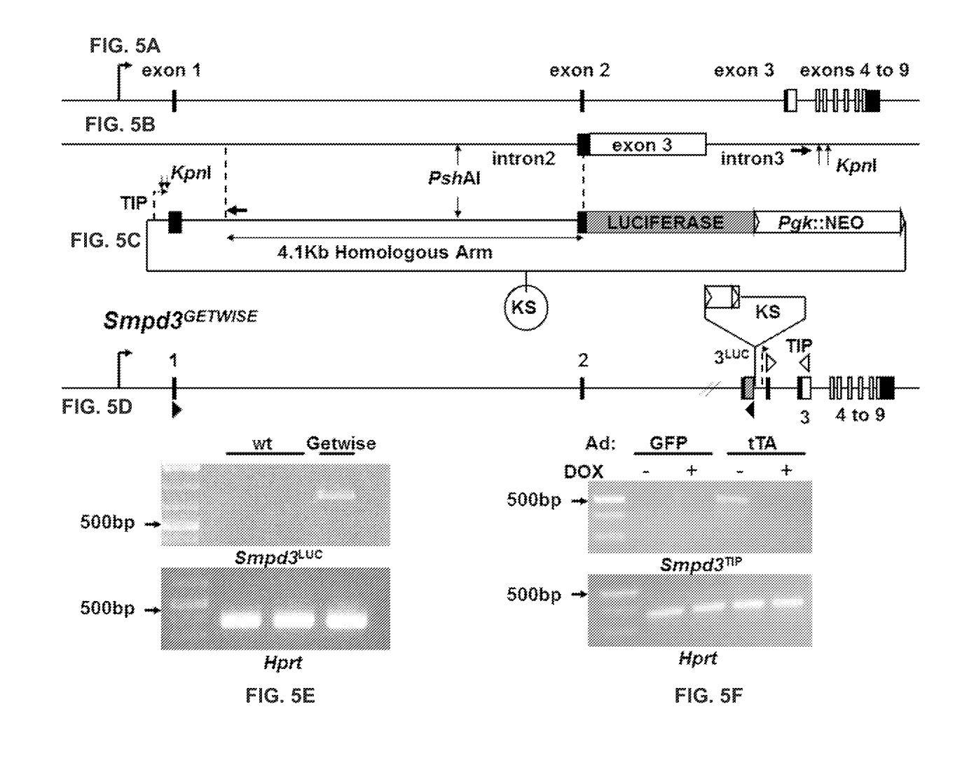

Ready-to-use GETWISE plasmids (pGETWISE), incorporating 6 GETWISE alleles from 5 target genes (Ppp1r12a, Ppp1r12a mutant, Ppp1r16b, Smpd3, Sgms1, and Ncrna00085) were produced, as illustrated in FIGS. 5, 6, 8, 9 and the Examples.

One GETWISE transgenic mouse line (Ppp1r16b) was developed and bred with endothelium-specific rtTA carrier (Teng, et al., Physiol Genomics, 11:99-107 (2002)) to confirm the ability of GETWISE allele to enable manipulation of gene expression in doxycycline-dependent and tissue-specific manner.

For two genes (Timap and Smpd3), GETWISE alleles were created by sequential cloning of DNA fragments; for 3 other genes (Mypt1, Sgms1, and Bachelor), pre-designed pGETWISE vectors were used. For Mypt1, both “classic” GETWISE allele and allele expressing constitutively active MYPT1 C/A were created. Out of 5 targeted genes, only Sgms1 was expressed in ES cells at a level allowing detection of homologous recombination with luciferase assay. In embryoid bodies derived from GETWISE clones, luciferase expression from Timap and Smpd3 alleles was confirmed. The analysis of embryoid bodies corroborated the fact that GETWISE clones express Timap and Smpd3 only in the presence of tTA and the absence of doxycycline.

### III. Methods of Making GETWISE Recombinant ES Cells

GETWISE recombinant embryonic stem (GETWISE ES) cells can be produced by introducing a linearized GETWISE vector into any ES cell and simply selecting homologously-recombined ES cell expressing the reporter gene. The strategy herein has been optimized to accelerate technical procedures from vector construction, to GETWISE ES cells screening. First, a reduced number of steps is needed to engineer DNA molecule for homologous recombination. Second, with GETWISE vectors, the homologous recombination itself occurs at higher frequency than with routinely used techniques. This diminishes the risk of failure and reduces time needed for the identification of positive clones.

Compared to the methods currently used to generate complete and conditional knockouts, GETWISE has several advantages, as discussed below.

First, the construction of the targeting vectors requires the cloning of only 1 or 2 DNA fragments, whereas the last generation vector for conditional knockout, knockout-first, requires cloning of 3 DNA fragments (Testa, et al., Genesis, 38:151-158 (2004)). Even with the introduction of recombineering method, cloning of 3 DNA fragments is still a lengthy procedure, and not without pitfalls (Bouvier, et al., Curr Protoc Mol Biol, Chapter 23:Unit 23 13 (2009)).

Second, the use of insertion vectors increases the frequency of homologous recombination thereby decreasing the amount of ES clones which has to be screened to detect positive clones (Adams, et al., Nat Genet, 36:867-871 (2004); Hasty, et al., Mol Cell Biol, 11:4509-4517 (1991)). Moreover, routinely used replacement vectors often render homologous recombination without generating a true conditional knockout; therefore, an additional screening step is necessary (Skarnes, et al., Nature, 474:337-342 (2011)). On the contrary, the GETWISE insertion vector warrants that all the recombinant clones are a true conditional knockout.

Third, for the genes expressed in GETWISE ES cells, the presence of the luciferase reporter allows pre-screening for homologous recombination using the robust luciferase assay. Although luciferase detection cannot be used as a sole screening method due to a possibility of luciferase expression in some ES clones with random cassette integration, pre-screening with the luciferase assay significantly decreases the amount of clones which have to be screened by PCR. For genes not expressed in GETWISE ES cells (no luciferase activity detected), but allowing gap creation in the vector, PCR is used as primary method to search for positive clones. A DNA pooling procedure which diminishes the number of PCR screening necessary for the identification of positive clones is described.

Using a mixture of constructs for co-electroporation is another possibility to reduce the number of clones to screen. These two approaches are especially useful for the genes with low to moderate rate of homologous recombination. Summarizing, features mentioned above accelerate technical steps from DNA manipulation to ES screening. Furthermore, they enhance the rate of success by diminishing the possibility that homologous recombination will never occur at the target site. Using pre-designed pGETWISE plasmids, targeted 4 genes were concurrently in ES cells within 1 month. Design of targeted vectors lasted 10 days, whereas gene targeting in ES cells and screening for gap repair took 19 days. If the structure of the targeted gene did not allow the creation of a gap, screening by inverse PCR would require additional 1-2 days.

A. Transfection

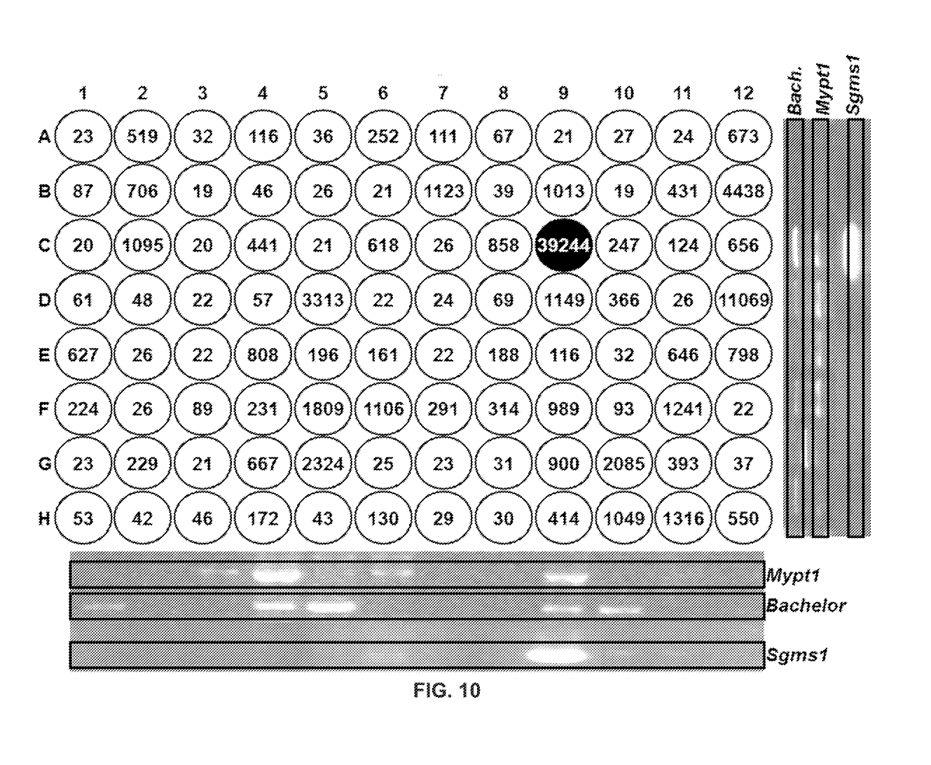

Methods for introducing the pGETWISE MYPT1C/A to ES cells, using electroporation, are disclosed. Using co-electroporation, a mixture of pGETWISE Mypt1 (wild-type), Sgms1, and Bachelor was introduced to ES cells. Co-electroporation was used to reduce the amount of ES clones to grow and screen for homologous recombination. For each experiment, 96 ES clones resistant to G418 were screened. The following clones were obtained: 2 ES clones carrying MYPT1C/A mutation (data not shown) and 4 homologous recombinant clones with “classic” Mypt1 allele, clones with Bachelor allele and 1 clone with Sgms1 allele (FIG. 10). DNA pooling allows researchers to reduce the number of initial screenings from 96 to 20. The luciferase assay identified a single positive clone at position C9 (ES clone recombinant for Sgms1) (FIG. 10). None of Mypt1 or Bachelor clones displayed luciferase activity, suggesting that, unlike Sgms1, these genes are not highly expressed in ES cells. These results were in accordance with a low number of gene-trap clones identified at Mypt1 and Bachelor loci (18 for Mypt1 and 21 for Bachelor comparing to 520 for Sgms1, www.informatics.jax.org).

B. Selection

Methods of pre-screening GETWISE vectors containing the luciferase reporter gene for positive ES clones using the luciferase assay are disclosed. In one method, the fast, inexpensive luciferase assay can be performed easily in 96-well format.

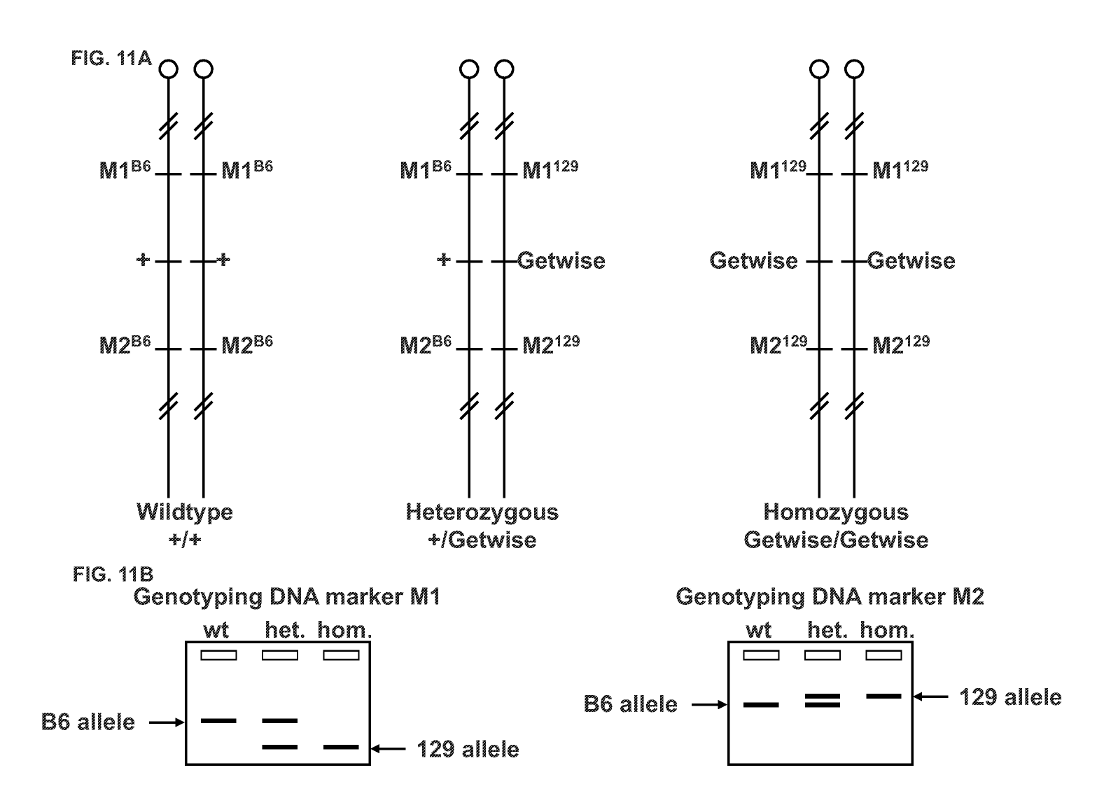

In certain embodiments, where homologous recombination of the insertion vector causes duplication at the integration site and there may be difficulty in distinguishing between homozygous and heterozygous GETWISE carriers by standard PCR, the genotype is inferred by following the segregation of at least 1 polymorphic DNA marker located within close proximity of the integration (FIGS. 11A-B).

### IV. Methods of Making GETWISE Transgenic Mice

A. GETWISE Principle

In the GETWISE system, an insertion vector is used instead of more traditional replacement vectors. Insertion vectors require a single reciprocal recombination, whereas replacement vectors require double recombination. Originally, homologous recombination with insertion vectors was shown to occur up to 92 fold more frequently than the recombination of replacement vector (Hasty, et al., Mol Cell Biol, 11:4509-4517 (1991)). Further studies analyzing homologous recombination at 29 loci showed 100% successful integration with a recombination frequency ranging from 5% to 82% (Adams, et al., Nat Genet, 36:867-871 (2004)). On the contrary, a high throughput homologous recombination project, with classical replacement vectors failed to detect homologous recombination in 368 out of 1,811 genes (-20%) (Skarnes, et al., Nature, 474:337-342 (2011)). Therefore, the use of insertion vectors seems to yield better results especially for the genes with low frequency of recombination (Cases, et al., Oncogene, 7:2525-2528 (1992); Koller, et al., Proc Natl Acad Sci USA, 88:10730-10734 (1991)).

The GETWISE system gives investigators a tremendous advantage to manipulate gene expression in a number of ways with the development of a single mouse strain. Although in some cases it requires a multi-step breeding to achieve the ability to express or silence gene in specific tissues, unlike transgenic mouse generation, breeding is a straightforward and simple procedure. The choice of tissue specificity does not have to be made at the moment of GETWISE animal generation, as specificity is achieved in later crosses. This gives GETWISE technique an unprecedented degree of versatility in comparison to the current techniques used for animal engineering.

Additionally, GETWISE enables investigator to study gene expression pattern using the same single mouse strain. All these features increase applicability of GETWISE and make GETWISE an attractive alternative to the current transgene/knockout generation methods.

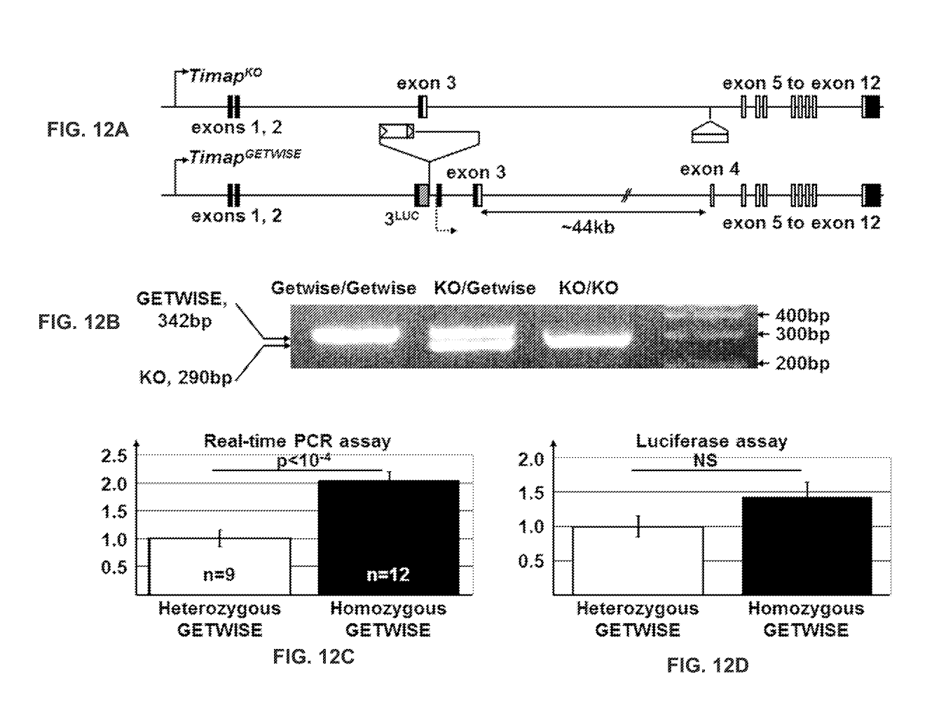

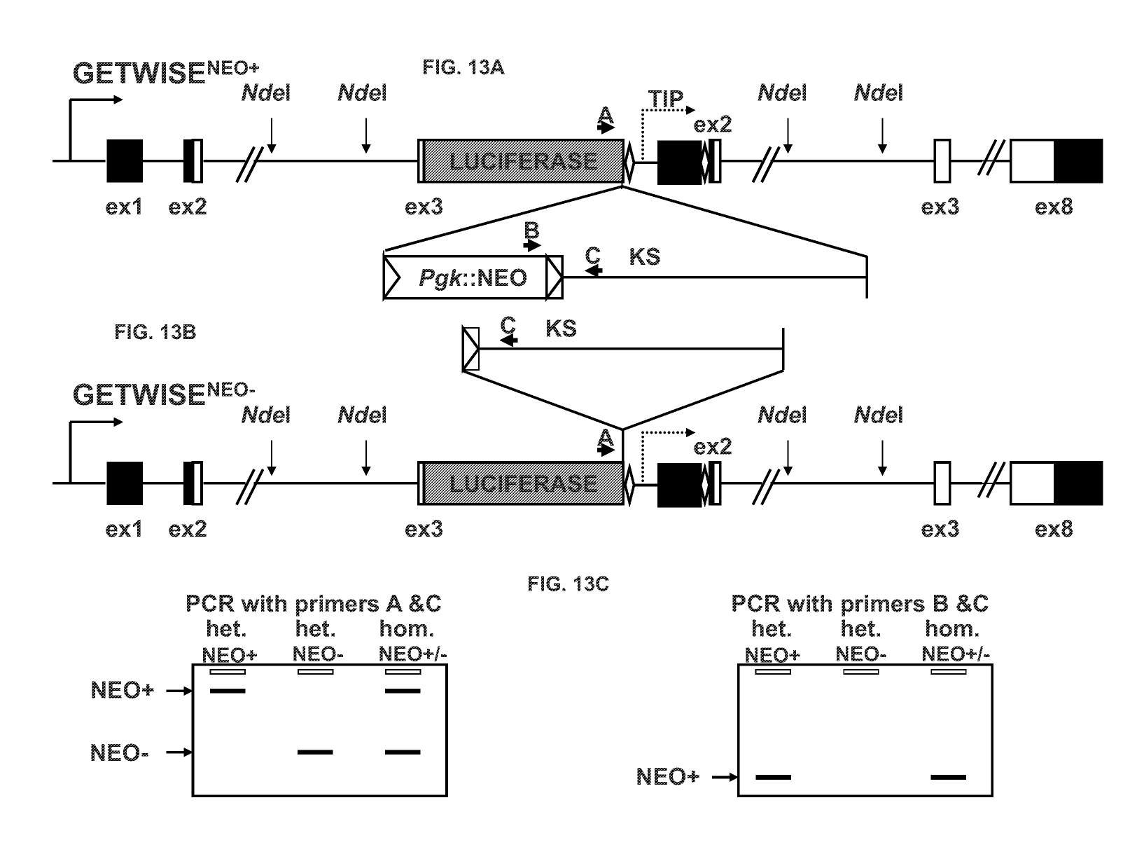

Methods of creating transgenic mice using GETWISE recombinant embryonic stem (GETWISE ES) cells are disclosed. The nuclei of GETWISE ES cells can be introduced into anucleated mouse oocytes to produce nuclear-transfusion mouse embryos, which can subsequently be implanted into a female mouse recipient. GETWISE transgenic mice can readily be identified by genotyping after birth (FIGS. 12 and 13).

B. Gene Silencing/Induction in GETWISE Mice

To further validate the techniques described herein, transgenic mice carrying TimapGETWISE allele were generated. Luciferase expression driven by Timap promoter was confirmed to correlate with Timap mRNA levels in vivo. These mice were employed to study the role of Timap transcriptional regulation in the model of acute lung injury (Poirier, et al., Respir Physiol Neurobiol, 179:334-337 (2011)). Furthermore, GETWISE/GETWISE animals were proved to no longer express Timap. The expression could be turned on in GETWISE/GETWISE offspring carrying tissue-specific rtTA upon an exposure to doxycyline. To compare the levels of inducible Timap expression to the levels of its endogenous expression, the amount of Timap mRNA in heterozygous TimapGETWISE animals was analyzed. Upon 5-day stimulation with 90 mg/kg/day doxycycline, the level of expression from TimapGETWISE allele was 1.5 fold higher than the level of expression from wild type allele. A higher or lower rate of inducible Timap expression can be achieved by increasing or lowering the dose of doxycycline. Altogether, these data proved the generation of a null allele of the targeted gene, and the possibility of doxycycline-dependent re-expression of the gene in the tissue of choice at the desired level.

In some embodiments, the control is reassigned over the gene transcription to the tetracycline-inducible promoter (TIP), which results in a ubiquitous loss of gene expression in the original animals. GETWISE animals enable the investigator to study the effect of gene knockout and to monitor the pattern of gene expression. For the ubiquitous or tissue-specific modulation of gene expression, investigator should set the crosses between GETWISE animals and tTA/rtTA-expressing animals. The choice of tissue does not have to be made before GETWISE animal is generated. As modulation of the gene expression in the offspring is triggered by the exposure to doxycycline, GETWISE technique has a build-in ability to circumvent problems such as early embryonic lethality due to gene deficiency or due to ectopic over-expression. The prior knowledge of the gene deficit or gene over-expression effects is not required and does not affect the way GETWISE animal is engineered.

C. Tissue-Specific Gene Expression in GETWISE Mice

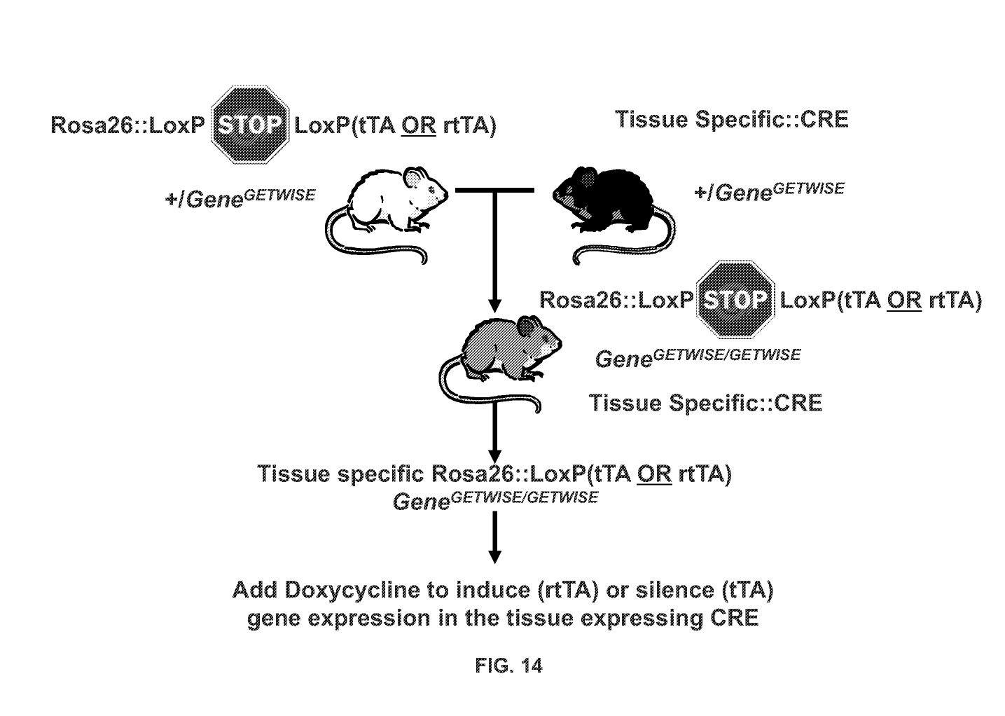

Methods for the tissue-specific expression of the transactivators are disclosed. Tissue-specific expression is achieved by breeding GETWISE mice with commercially available transgenic mice expressing CRE recombinase in a tissue-specific pattern. A large number of commercially available tissue-specific CRE transgenic mice allows investigators to use GETWISE to achieve a doxycycline-dependent control of gene expression in a larger variety of tissues (FIG. 14).

Methods to circumvent the limited choice of tissue-specific tTA/rtTA expressing mice are also disclosed. To expand the choice of tissues with tTA/rtTA expression, the control over transactivators expression is established under CRE. To achieve this control, GETWISE carriers are first crossed with mice expressing tTA/rtTA under the ubiquitous Rosa26 promoter. In the resulting mice, heterozygous for both GETWISE and Rosa26 transgenes (white mouse), tTA/rtTA expression is halted by the presence of the LoxP-STOP-LoxP cassette. To remove this cassette and enable a tissue-specific expression of transactivators, white mice are next crossed with another double heterozygous mice carrying GETWISE and CRE transgene (black mouse). Among the offspring of the white and black mice, homozygous GETWISE/GETWISE mice are selected. These mice are genotyped for the presence of tTA/rtTA and CRE transgenes. Those GETWISE/GETWISE mice, heterozygous for both transactivators and CRE transgenes, have a deletion of the LoxP-STOP-LoxP cassette in the tissues expressing CRE. This deletion causes the expression of the transactivator tTA or rtTA; exposure to doxycycline induces (rtTA) or silences (tTA) expression of the targeted gene from GETWISE allele in the tissues expressing CRE recombinase.

In certain embodiments, where homologous recombination of the insertion vector causes duplication at the integration site during large litter genotyping, real-Time PCR can be used to distinguish mice with 2 copies of the vector (homozygous) from the mice with 1 copy (heterozygous) (FIG. 12).

One of skill in the art will appreciate that, where homologous recombination of the insertion vector causes duplication at the integration site amongst mice from whom the ex-vivo utilization of the specific tissue is of interest, the luciferase assay can be used to distinguish mice with 2 copies of the vector (homozygous) from the mice with 1 copy (heterozygous) (FIG. 12).

If basal TIP activity is observed in GETWISE vectors due to the activity of tetracycline-responsive elements in the absence of proper stimulation and basal transcriptional activity from TIP impedes the generation of animals with the complete loss of expression, one of skill in the art will appreciate that, TIP can be placed between 2 FRT sites, allowing the deletion of TIP after breeding with commercially available transgenic mice expressing FLP recombinase in the germline (www.jax.org).

In certain other embodiments, where basal TIP activity is observed in GETWISE vectors due to the activity of tetracycline-responsive elements in the absence of proper stimulation and basal transcriptional activity from TIP impedes the generation of animals with the complete loss of expression, basal transcriptional activity is abrogated by breeding to the transgenic mice expressing tTS, a chimeric molecule containing the tetracycline repressor and a transcriptional repressing domain (Mallo. et al., Genomics, 81:356-360 (2003)).

In certain other embodiments, where an integration of the entire targeting vector, including the pBluescript sequences, interferes with the expression from the TIP, the new generation of pGETWISE plasmids which carry a loxP site directly upstream from the first FRT site, are employed (FIG. 6). Where an integration of the entire targeting vector, including the pBluescript sequences, interferes with the expression from the TIP in the mutant mice, the neomycine cassette as well as the entire pBluescript sequence are remove through CRE-mediated recombination.

One of skill in the art will appreciate that, where there is a relative shortage of transgenic mice which express tTA or rtTA in a tissue-specific manner, the use of 2 transgenic mice (Gt(ROSA)26Sortm1 (rtTA,EGFP)Nagy/J and Gt(ROSA)26Sortm1 (tTA)Roos/J), which conditionally express rtTA and tTA transactivators from the ubiquitous Rosa26 promoter, are employed. (Zambrowicz, et al., Proc Natl Acad Sci USA, 94:3789-3794 (1997)).

### EXAMPLES

Methods and Materials

### Primers

A list of primers can be found in Table 1. The primers used in this application are indicated in the table, with their sequence, their position in the gene, and the size of the amplicon. For primer #9, the junction between exon 1 and exon 3 is indicated by an arrow. For primer #29, the 11 nt corresponding to the 5′ end of Mypt1 intron 1 sequence are underlined.

TABLE 1
Primers

| SEQ ID | | | Position in the | Size of |
|---|---|---|---|---|
| NO | Gene | Sequence (5′ to 3′) | gene | amplicon |
| 1 | Timap | AAGGTACCGGAGAGTTCAAC | Homologous | 4,518 bp |
| | | ACGGATGG | arm (intron 2) | |
| 2 | Timap | TTAAGCTTCCTGCCCTTGCT | Homologous | 4,518 bp |
| | | CTGCCTGG | arm (axon 3) | |
| 3 | | GGAAACAGCTATGACCATG | Primer in | 1,988 bp |
| | | | bluescript | (Round 1) |
| | | | backbone | |
| 4 | Timap | GAGCATATGGGATTGAAAGG | Primer in the | 1,988 bp |
| | | | ApaI gap, ES | (Round 1) |
| | | | screening | |
| 5 | | AAATTAACCCTCACTAAAGG | Primer in | 1,928 bp |
| | | | bluescript | (Round 2) |
| | | | backbone | |
| 6 | Timap | ACCAACACACAGTGTCATGG | Primer in the | 1,928 bp |
| | | | ApaI gap, ES | (Round 2) |
| | | | screening | |
| 7 | Timap | CAGAATCAAGCTCAACGTGG | Forward primer | 757 bp |
| | | | in exon 2 | |
| 8 | Luciferase | TCCCTGTCGAAGGACTCTGG | Reverse primer | 757 bp |
| | | | in luciferase | |
| 9 | Timap | CCAACCAGAG_GCCCCACCAT | Forward primer | 389 bp |
| | | | exon 1/3 | |
| 10 | Timap | GGCTAACCTTGTTCTTCAGG | Reverse primer | 389 bp |
| | | | in exon 4 | |
| 11 | Capn1 | AAACGCCATCAAGTACCTGG | Forward primer | 303 bp |
| | | | in exon 2 | |
| 12 | Capn1 | TCGGTGCAGAATAGTCTCGTT | Reverse primer | 303 bp |
| | | | in exon 4 | |
| 13 | Timap | CCCTGATTTGTGCAATGAGG | Forward primer | 257 bp |
| | | | in exon 4 | |
| 14 | Timap | TGGGTTCATCCTCGCAGAGG | Reverse primer | 257 bp |
| | | | in exon 6 | |
| 15 | Hprt | TCATGCCGACCCGCAGTCCC | Forward primer | 373 bp |
| | | | in exon 1 | |
| 16 | Hprt | GTTAAGAGATCATCTCCACC | Reverse primer | 373 bp |
| | | | in exon 4 | ( |
| 17 | Smpd3 | GGTACCCTCTTCTTGCATCTG | Homologous | 4,108 bp |
| | | TCTGTGAC | arm (intron 2) | |
| 18 | Smpd3 | AGATCTGTGACTCTGGATGAT | Homologous | 4,108 bp |
| | | CAGTGGC | arm (exon 3) | |
| 19 | Smpd3 | GCCCAGGAGTCAGGCTAACC | In intron 2 of | 1,455 bp |
| | | | Smpd3, ES | (Round 1) |
| | | | screening | |
| 20 | Smpd3 | ACACACTCAGATGTCTCACC | In intron 3 of | 1,455 bp |
| | | | Smpd3, ES | (Round 1) |
| | | | screening | |
| 21 | Smpd3 | TTGGGGTCTTCACAGGTGCC | In intron 2 of | 1,138 bp |
| | | | Smpd3, ES | (Round 2) |
| | | | screening | |
| 22 | Smpd3 | GGGTTCCCAGCTCGCCAGCC | In intron 3 of | 1,138 bp |
| | | | Smpd3, ES | (Round 2) |
| | | | screening | |
| 23 | Smpd3 | CCGCGAGAGCCGTCTCTAGG | in exon 1 used | 889 bp |
| | | | with #8 | |
| 24 | Timap | AGCCTCGGGCACATGAAAGG | in exon of TIP | 612 bp |
| 25 | Smpd3 | GAACCCGAGAAAGGCAAAGG | in exon 3 of | 612 bp |
| | | | Smpd3 | |
| 26 | Mypt1 | TGTTAACTGTACCTTTGCGG | Homologous | 5,756 bp |
| | | | arm (intron 1) | |
| 27 | Mypt1 | CCACAGGAAGCAGCTGCATGG | Homologous | 5,756 bp |
| | | | arm (exon 2) | |
| 28 | Mypt1 | AATTAATTAAGCTCGCGATAA | In exon 1 of | 283 bp |
| | | GAGGAGCC | Mypt1 | |
| 29 | Mypt1 | AATTAATTAAGGCACACAGACC | Junction exon | 283 bp |
| | | TGGTGCAGGGCGGTGAGTCC | 1/ intron 1 of | |
| | | | Mypt1 | |
| 30 | Mypt1 | TTAATTAAATGCAGATGGCGGA | Primer for | 1,133 bp |
| | | CGCGAA | Mypt1 mutant | |
| | | | cDNA | |
| 31 | Mypt1 | TTAATTAATTTTATTAGGAAAG | Primer for | 1,133 bp |
| | | GACAGT | Mypt1 mutant | |
| | | | cDNA | |
| 32 | Mypt1 | CCCTTTGTAGCACTGGTTGG | Primer in the | 2,655 bp |
| | | | AflII gap, ES | (Round 1) |
| | | | screening | |
| 33 | Luciferase | CAATTGTTCCAGGAACCAGG | Primer in | 2,655 bp |
| | | | Luciferase | (Round 1) |
| | | | sequence | |
| 34 | Mypt1 | GTCAAGACTATAAGTACAGG | Primer in the | 2,542 bp |
| | | | AflII gap, ES | (Round 2) |
| | | | screening | |
| 35 | Luciferase | GCGGTTCCATCTTCCAGCGG | Primer in | 2,542 bp |
| | | | Luciferase | (Round 2) |
| | | | sequence | |
| 36 | Bachelor | GATATCTGATGAGAGGAGCTA | Homologous | 4,071 bp |
| | | CCTGC | arm,(exon 2) | |
| 37 | Bachelor | GTCCTCTTCAATTTTGCAGC | Homologous | 4,071 bp |
| | | | arm (exon 3 | |
| 38 | Bachelor | GGGTTGGGAGGAGTAGGAGG | Primer in the | 1,040 bp |
| | | | gap used with | (Round 1) |
| | | | #33 | |
| 39 | Bachelor | GGTTTTGAAGATAGGAGCGG | Primer in the | 958 bp |
| | | | gap used with | (Round 2) |
| | | | #35 | |
| 40 | Sgms1 | CTAGACACTATTCCAGATGG | Homologous | 4,979 bp |
| | | | arm (intron 5) | |
| 41 | Sgms1 | CACTATCCCTCCTTGGCTGG | Homologous | 4,979 bp |
| | | | arm,(exon 6 | |
| 42 | Sgms1 | TCATTCTAGAATCAGACTGG | Primer in the | 2,157 bp |
| | | | gap used with | (Round 1) |
| | | | #33 | |
| 43 | Sgms1 | CTAGTTGGAGAGAAGAGCGG | Primer in the | 2,066 bp |
| | | | gap used with | (Round 2) |
| | | | #35 | |

### Plasmids

The pGKfloxNEO plasmid was obtained from Pr. Richard Behringer (UTMD, Houston, Tex.). pRevTRE, pGL3-basic, and pEF-DEST51 plasmids were purchased from CLONTECH, PROMEGA and INVITROGEN. The genomic sequences were amplified from BAC clones by PCR with Supertaq enzyme (AMBION) and cloned into pCR4 (INVITROGEN) for Smpd3 and Timap or pCR8/GW (INVITROGEN). The transfer of the homologous region from pCR8/GW to pGETWISE plasmids was achieved through DNA recombination with LRII clonase enzyme (INVITROGEN).

To create the internal gap, plasmids were digested overnight with the appropriate enzyme. After self-ligation, the plasmid was introduced into competent E. coli TOP10 strain (INVITROGEN) by heat shock. Clones carrying the internal deletion were identified by restriction analysis and PCR. Smpd3 and Timap targeting constructs. The tetracycline inducible promoter (TIP) consists of 540 by containing the tetracycline responsive element from pRevTRE (position 2768 to 3308), 212 by of the entire Timap exon1, and 146 by of the 5′ end sequence from Timap intron1. The homologous regions were cloned at KpnI-BglII (Smpd3) or KpnI-HindIII (Timap) sites of pGL3. Then the KpnI-SalI fragments were sub-cloned into KpnI-SalI sites of pGKfloxNEO plasmid. These plasmids were linearized at KpnI and blunt ended to introduce the TIP cassette. For ApaI gapping, Timap targeting vector was transferred to the dam- dcm- E. coli strain ER2925 (NEW ENGLAND BIOLABS) to allow de-methylation of the ApaI sites.

pGETWISE Plasmids Engineering

KpnI-PacI-AscI-LUCIFERASE-PvuII cassette was cloned into KpnI-EcoRV sites of pGKfloxNEO as KpnI-PvuII insert, yielding intermediate plasmid VEC1.

The sequence of the KpnI-PacI-AscI-LUCIFERASE-PvuII cassette (SEQ ID NO: 44) is shown below:

GGTACCTTAATTAAGGCGCGCCGGTACTGTTGGTAAAGCCAC
CATGGAAGACGCCAAAAACATAAAGAAAGGCCCGGCGCCATTCTATCCGC
TGGAAGATGGAACCGCTGGAGAGCAACTGCATAAGGCTATGAAGAGATAC
GCCCTGGTTCCTGGAACAATTGCTTTTACAGATGCACATATCGAGGTGGA
CATCACTTACGCTGAGTACTTCGAAATGTCCGTTCGGTTGGCAGAAGCTA
TGAAACGATATGGGCTGAATACAAATCACAGAATCGTCGTATGCAGTGAA
AACTCTCTTCAATTCTTTATGCCGGTGTTGGGCGCGTTATTTATCGGAGT
TGCAGTTGCGCCCGCGAACGACATTTATAATGAACGTGAATTGCTCAACA
GTATGGGCATTTCGCAGCCTACCGTGGTGTTCGTTTCCAAAAAGGGGTTG
CAAAAAATTTTGAACGTGCAAAAAAAGCTCCCAATCATCCAAAAAATTAT
TATCATGGATTCTAAAACGGATTACCAGGGATTTCAGTCGATGTACACGT
TCGTCACATCTCATCTACCTCCCGGTTTTAATGAATACGATTTTGTGCCA
GAGTCCTTCGATAGGGACAAGACAATTGCACTGATCATGAACTCCTCTGG
ATCTACTGGTCTGCCTAAAGGTGTCGCTCTGCCTCATAGAACTGCCTGCG
TGAGATTCTCGCATGCCAGAGATCCTATTTTTGGCAATCAAATCATTCCG
GATACTGCGATTTTAAGTGTTGTTCCATTCCATCACGGTTTTGGAATGTT
TACTACACTCGGATATTTGATATGTGGATTTCGAGTCGTCTTAATGTATA
GATTTGAAGAAGAGCTGTTTCTGAGGAGCCTTCAGGATTACAAGATTCAA
AGTGCGCTGCTGGTGCCAACCCTATTCTCCTTCTTCGCCAAAAGCACTCT
GATTGACAAATACGATTTATCTAATTTACACGAAATTGCTTCTGGTGGCG
CTCCCCTCTCTAAGGAAGTCGGGGAAGCGGTTGCCAAGAGGTTCCATCTG
CCAGGTATCAGGCAAGGATATGGGCTCACTGAGACTACATCAGCTATTCT
GATTACACCCGAGGGGGATGATAAACCGGGCGCGGTCGGTAAAGTTGTTC
CATTTTTTGAAGCGAAGGTTGTGGATCTGGATACCGGGAAAACGCTGGGC
GTTAATCAAAGAGGCGAACTGTGTGTGAGAGGTCCTATGATTATGTCCGG
TTATGTAAACAATCCGGAAGCGACCAACGCCTTGATTGACAAGGATGGAT
GGCTACATTCTGGAGACATAGCTTACTGGGACGAAGACGAACACTTCTTC
ATCGTTGACCGCCTGAAGTCTCTGATTAAGTACAAAGGCTATCAGGTGGC
TCCCGCTGAATTGGAATCCATCTTGCTCCAACACCCCAACATCTTCGACG
CAGGTGTCGCAGGTCTTCCCGACGATGACGCCGGTGAACTTCCCGCCGCC
GTTGTTGTTTTGGAGCACGGAAAGACGATGACGGAAAAAGAGATCGTGGA
TTACGTCGCCAGTCAAGTAACAACCGCGAAAAAGTTGCGCGGAGGAGTTG
TGTTTGTGGACGAAGTACCGAAAGGTCTTACCGGAAAACTCGACGCAAGA
AAAATCAGAGAGATCCTCATAAAGGCCAAGAAGGGCGGAAAGATCGCCGT
GTAATTCTAGAGTCGGGGCGGCCGGCCGCTTCGAGcagacatgataagat
acattgatgagtttggacaaaccacaactagaatgcagtgaaaaaaatga
ttatttgtgaaatttgtgatgctattgctttatttgtaaccattataagc
tgcaataaacaagttaacaacaacaattgcattcattttatgtttcaggt
tcagggggaggtgtgggaggttttttaaagcaagtaaaacctctacaaat
gtggtaAAATCGATAAGCAGCTG

The luciferase coding sequence is italicized. The SV40 late polyA signal is highlighted in lower cases. The luciferase cassette was amplified from pGL3-basic (PROMEGA) by PCR. For cloning purposes, linkers (highlighted in bold) were added at the end of the cassette (KpnI (GGTACC, SEQ ID NO: 45), PacI (TTAATTAA, SEQ ID NO: 46), AscI (GGCGCGCC, SEQ ID NO: 47), and PvuII (CAGCTG, SEQ ID NO: 48)). The resulting KpnI-PacI-AscI-LUCIFERASE-PvuII cassette (shown) was cloned into KpnI-EcoRV sites of pGKfloxNEO as KpnI-PvuII insert, yielding intermediate plasmid VEC1. VEC1 was used to create the next plasmid carrying TIP.

Sequences of FRT-TIP-FRT cassettes are also disclosed. FRT-TetOCMVex1-in1 (partial)-FRT and FRT-TetOCMVex1 (partial)-FRT cassettes were cloned as PvuI-AscI insert into PacI-AscI sites of VEC1, yielding intermediate plasmids VEC2a and VEC2b.

The sequence of the FRT-TetOCMVex1-in1 (partial)-FRT cassette (SEQ ID NO: 49) is given below:

CGATCGGAAGTTCCTATACTTTCTAGAGAATAGGAACTTCtcgg
gtgatctgactgatcccgcagattggagatcgccgcccgtgcctgccgat
tgggtgcagatctcgagtttaccactccctatcagtgatagagaaaagtg
aaagtcgagtttaccactccctatcagtgatagagaaaagtgaaagtcga
gtttaccactccctatcagtgatagagaaaagtgaaagtcgagtttacca
ctccctatcagtgatagagaaaagtgaaagtcgagtttaccactccctat
cagtgatagagaaaagtgaaagtcgagtttaccactccctatcagtgata
gagaaaagtgaaagtcgagtttaccactccctatcagtgatagagaaaag
tgaaagtcgagctcggtacccgggtcgagtaggcgtgtacggtgggaggc
ctatataagcagagctcgtttagtgaaccgtcagatcgcctggagacgcc
atccacgctgttttgacctccatagaagacaccgggaccgatccagcctc
cgcggccccgaattcgagctcggtacccggggatctACAGCTCTTGATGG
AAACCTTCTGCAGAGAACAATGGCCCCTCTGGGTTATTTTAAACTTCACT
CTAAGCCTTCTCCACCTGCTCTGCTGTCTCTAATTGCTCCAGAAACAGCG
TCATTCACAACTGGATTATTAATGAACTGAGCGGGGAGCCTCGGGCACAT
GAAAGGGGACTTCAGACTTGGGGAAGTCTTCGAAGGACCCAACCAGAGGT
GGGTTTTGTTGTTTTAAGACAAGGTCTCATGTGGCACAGACAGGCTGGCC
TCAAATCCATAAGTATGCCTGAGAGCTGGATTTACATGTGTTTGTTACCA
CACCTGGTACTGGCACACATGCAGGGTACTGAAGTTCCTATACTTTCTAG
AGAATAGGAACTTCGGCGCGCC

The sequence of the FRT-TetOCMVex1 (partial)-FRT cassette (SEQ ID NO: 50) is given below:

CGATCGGAAGTTCCTATACTTTCTAGAGAATAGGAACTTCtcgg
gtgatctgactgatcccgcagattggagatcgccgcccgtgcctgccgat
tgggtgcagatctcgagtttaccactccctatcagtgatagagaaaagtg
aaagtcgagtttaccactccctatcagtgatagagaaaagtgaaagtcga
gtttaccactccctatcagtgatagagaaaagtgaaagtcgagtttacca
ctccctatcagtgatagagaaaagtgaaagtcgagtttaccactccctat
cagtgatagagaaaagtgaaagtcgagtttaccactccctatcagtgata
gagaaaagtgaaagtcgagtttaccactccctatcagtgatagagaaaag
tgaaagtcgagctcggtacccgggtcgagtaggcgtgtacggtgggaggc
ctatataagcagagctcgtttagtgaaccgtcagatcgcctggagacgcc
atccacgctgttttgacctccatagaagacaccgggaccgatccagcctc
cgcggccccgaattcgagctcggtacccggggatctACAGCTCTTGATGG
AAACCTTCTGCAGAGAACAATGGCCCCTCTGGGTTATTTTAAACTTCACT
CTAAGCCTTCTCCACCTGCTCTGCTGTCTCTAATTGCTCCAGAAACAGCG
TCATTCACAACTGGATTATTAATGAACTGAGCGGGGAGCCTCGGGCACAT
GAAAGGGGACTTCAGACTTGGGGAAGTTAATTAAGAAGTTCCTATACTTT
CTAGAGAATAGGAACTTCGGCGCGCC

The tetracycline responsive elements, originated from pREV-TRE, are highlighted in lower cases. The TIP cassettes were amplified by PCR from pGETWISE-Timap plasmid with primers containing FRT site at their 5′ end. The last t at the end of the tetracycline responsive elements had been mutated from C for molecular cloning reasons. The first exon (non-coding) from Timap gene is italicized. The partial first intron (5′ end) from Timap gene is in bold font. The flanking FRT sites are underlined. The FRT sites were shown to be functional by in vitro assays (data not shown). For cloning purposes, linkers (highlighted in bold) were added at the end of the cassettes (PvuI (CGATCG, SEQ ID NO: 51), AscI (GGCGCGCC, SEQ ID NO: 47), and PacI (TTAATTAA, SEQ ID NO: 46)). The resulting FRT-TetOCMVex1-in1 (partial)-FRT (SEQ ID NO: 49) and FRT-TetOCMVex1 (partial)-FRT (SEQ ID NO: 50) cassettes were cloned as PvuI-AscI insert into PacI-AscI sites of VEC1, yielding intermediate plasmids VEC2a and VEC2b.

Sequences of the DEST cassettes with (+) orientation (SEQ ID NO: 52) and (−) orientation are disclosed. The sequence of the DEST cassette with (+) orientation (SEQ ID NO: 52) is given below:

GGCGCGCCCAAGCTGGCTAGGTAAGCTTGATCAACAAGTTTGT
ACAAAAAAGCTGAACGAGAAACGTAAAATGATATAAATATCAATATATTA
AATTAGATTTTGCATAAAAAACAGACTACATAATACTGTAAAACACAACA
TATCCAGTCACTATGGCGGCCGCATTAGGCACCCCAGGCTTTACACTTTA
TGCTTCCGGCTCGTATAATGTGTGGATTTTGAGTTAGGATCCGGCGAGAT
TTTCAGGAGCTAAGGAAGCTAAAatggagaaaaaaatcactggatatacc
accgttgatatatcccaatggcatcgtaaagaacattttgaggcatttca
gtcagttgctcaatgtacctataaccagaccgttcagctggatattacgg
cctttttaaagaccgtaaagaaaaataagcacaagttttatccggccttt
attcacattcttgcccgcctgatgaatgctcatccggaattccgtatggc
aatgaaagacggtgagctggtgatatgggatagtgttcacccttgttaca
ccgttttccatgagcaaactgaaacgttttcatcgctctggagtgaatac
cacgacgatttccggcagtttctacacatatattcgcaagatgtggcgtg
ttacggtgaaaacctggcctatttccctaaagggtttattgagaatatgt
ttttcgtctcagccaatccctgggtgagtttcaccagttttgatttaaac
gtggccaatatggacaacttcttcgcccccgttttcaccatgggcaaata
ttatacgcaaggcgacaaggtgctgatgccgctggcgattcaggttcatc
atgccgtctgtgatggcttccatgtcggcagaatgcttaatgaattacaa
cagtactgcgatgagtggcagggcggggcgTAAAGATCTGGATCCGGCTT
ACTAAAAGCCAGATAACAGTATGCGTATTTGCGCGCTGATTTTTGCGGTA
TAAGAATATATACTGATATGTATACCCGAAGTATGTCAAAAAGAGGTGTG
CTATGAAGCAGCGTATTACAGTGACAGTTGACAGCGACAGCTATCAGTTG
CTCAAGGCATATATGATGTCAATATCTCCGGTCTGGTAAGCACAACCATG
CAGAATGAAGCCCGTCGTCTGCGTGCCGAACGCTGGAAAGCGGAAAATCA
GGAAGGGATGGCTGAGGTCGCCCGGTTTATTGAAATGAACGGCTCTTTTG
CTGACGAGAACAGGGACTGGTGAAATGCAGTTTAAGGTTTACACCTATAA
AAGAGAGAGCCGTTATCGTCTGTTTGTGGATGTACAGAGTGATATTATTG
ACACGCCCGGGCGACGGATGGTGATCCCCCTGGCCAGTGCACGTCTGCTG
TCAGATAAAGTCTCCCGTGAACTTTACCCGGTGGTGCATATCGGGGATGA
AAGCTGGCGCATGATGACCACCGATATGGCCAGTGTGCCGGTCTCCGTTA
TCGGGGAAGAAGTGGCTGATCTCAGCCACCGCGAAAATGACATCAAAAAC
GCCATTAACCTGATGTTCTGGGGAATATAAATGTCAGGCTCCGTTATACA
CAGCCAGTCTGCAGGTCGACCATAGTGACTGGATATGTTGTGTTTTACAG
TATTATGTAGTCTGTTTTTTATGCAAAATCTAATTTAATATATTGATATT
TATATCATTTTACGTTTCTCGTTCAGCTTTCTTGTACAAAGTGGTTGATC
TAGAGGGCCCGCGGTTCGAAGGTAAGCCTAGGCGCGCC

The sequence of the DEST cassette with (−) orientation (SEQ ID NO: 53), is given below:

GGCGCGCCTAGGCTTACCTTCGAACCGCGGGCCCTCTAGATC
AACCACTTTGTACAAGAAAGCTGAACGAGAAACGTAAAATGATATAAATA
TCAATATATTAAATTAGATTTTGCATAAAAAACAGACTACATAATACTGT
AAAACACAACATATCCAGTCACTATGGTCGACCTGCAGACTGGCTGTGTA
TAACGGAGCCTGACATTTATATTCCCCAGAACATCAGGTTAATGGCGTTT
TTGATGTCATTTTCGCGGTGGCTGAGATCAGCCACTTCTTCCCCGATAAC
GGAGACCGGCACACTGGCCATATCGGTGGTCATCATGCGCCAGCTTTCAT
CCCCGATATGCACCACCGGGTAAAGTTCACGGGAGACTTTATCTGACAGC
AGACGTGCACTGGCCAGGGGGATCACCATCCGTCGCCCGGGCGTGTCAAT
AATATCACTCTGTACATCCACAAACAGACGATAACGGCTCTCTCTTTTAT
AGGTGTAAACCTTAAACTGCATTTCACCAGTCCCTGTTCTCGTCAGCAAA
AGAGCCGTTCATTTCAATAAACCGGGCGACCTCAGCCATCCCTTCCTGAT
TTTCCGCTTTCCAGCGTTCGGCACGCAGACGACGGGCTTCATTCTGCATG
GTTGTGCTTACCAGACCGGAGATATTGACATCATATATGCCTTGAGCAAC
TGATAGCTGTCGCTGTCAACTGTCACTGTAATACGCTGCTTCATAGCACA
CCTCTTTTTGACATACTTCGGGTATACATATCAGTATATATTCTTATACC
GCAAAAATCAGCGCGCAAATACGCATACTGTTATCTGGCTTTTAGTAAGC
CGGATCCAGATCTTTAcgccccgccctgccactcatcgcagtactgttgt
aattcattaagcattctgccgacatggaagccatcacagacggcatgatg
aacctgaatcgccagcggcatcagcaccttgtcgccttgcgtataatatt
tgcccatggtgaaaacgggggcgaagaagttgtccatattggccacgttt
aaatcaaaactggtgaaactcacccagggattggctgagacgaaaaacat
attctcaataaaccattagggaaataggccaggttttcaccgtaacacgc
cacatcttgcgaatatatgtgtagaaactgccggaaatcgtcgtggtatt
cactccagagcgatgaaaacgtttcagtttgctcatggaaaacggtgtaa
caagggtgaacactatcccatatcaccagctcaccgtctttcattgccat
acggaattccggatgagcattcatcaggcgggcaagaatgtgaataaagg
ccggataaaacttgtgcttatttttctttacggtctttaaaaaggccgta
atatccagctgaacggtctggttataggtacattgagcaactgactgaaa
tgcctcaaaatgttctttacgatgccattgggatatatcaacggtggtat
atccagtgatttttttctccatTTTAGCTTCCTTAGCTCCTGAAAATCTC
GCCGGATCCTAACTCAAAATCCACACATTATACGAGCCGGAAGCATAAAG
TGTAAAGCCTGGGGTGCCTAATGCGGCCGCCATAGTGACTGGATATGTTG
TGTTTTACAGTATTATGTAGTCTGTTTTTTATGCAAAATCTAATTTAATA
TATTGATATTTATATCATTTTACGTTTCTCGTTCAGCTTTTTTGTACAAA
CTTGTTGATCAAGCTTACCTAGCCAGCTTGGGCGCGCC

The DEST cassette was amplified from pEFDEST51 by PCR. Linkers, containing the AscI restriction site (GGCGCGCC, SEQ ID NO: 47), added at each end of the cassette for cloning purposes, are highlighted in bold. The attR1 site is italicized and underlined. The Chloramphenicol resistance gene is highlighted in lower case. The ccdB gene is underlined. The attR2 site is italicized. The DEST cassette, in both orientation (+ and −), was cloned at the AscI site of VEC2a and VEC2b, yielding pGETWISE1.1 (+ and −) and pGETWISE2.1 (+ and −). pGETWISE plasmids were propagated in E. coli ccd survival strain (INVITROGEN) because of the presence of the lethal ccdB gene in the DEST cassette.

DEST cassette from pEF-DEST51, in both orientation (+ and −), was cloned at AscI site of VEC2a and VEC2b, yielding pGETWISE1.1 (+ and −) and pGETWISE2.1 (+ and −). pGETWISE plasmids were propagated in E. coli ccd survival strain (INVITROGEN) because of the presence of the lethal ccd gene in DEST cassette.

### ES Culture

J1 ES cells (Li, et al., Cell, 69:915-926 (1992)) were purchased from OPEN BIOSYSTEMS at passage 11. ES cells were grown on mitomycin-arrested mouse feeder layers. Electroporation was performed using either Gene Pulser II (BIORAD) or AMAXA nucleofector II (LONZA). With Gene Pulser II (Timap, and Smpd3), 7×10⁶ cells were mixed with 25 μg linearized plasmid and electroporated into a 4 mm gap cuvette at 500 μF ad 250 V. With AMAXA (Mypt1, Bachelor, and Sgms1), a mouse ES cell nucleofector kit was used according to the manufacturer's instructions; electroporation was performed with program A23. Selection with G418 (350 μg/ml) was initiated 24 hours after electroporation. After 10 to 12 days of selection, individual ES clones were transferred to 96-well plates and maintained growing on feeder layers. After 3 days, cells in the original plate were split to 3 additional plates without feeder layer. The original plate was frozen at −80° C. The clones in the additional plates were grown for 3 more days, then 2 plates were subjected to DNA isolation and 1 plate was extracted for luciferase assay. Clones positive for homologous recombination were thawed, and expanded on feeder layer in 12-well plate. After 4 days, the cells were split to collect DNA and freeze 2 cryotubes for long term storage in liquid nitrogen. DNA was used to confirm the homologous recombination.

### DNA Preparation

ES clones grown in wells of 96-well plates were lysed in 100 μl of lysis buffer (10 mM Tris-HCl pH8, 1% SDS, 5 mM EDTA, 5 mM NaCl, 2 μg proteinase K) at 60° C. for 3 h. To reduce the cost and time of PCR screening, cell lysates from 96-well plates were analyzed in pools. In the first 96-well plate, clones in every column were pooled together. In the second plate, clones in every row were pooled together. DNA was extracted with phenol/chloroform and precipitated with ethanol. After washing with 70% ethanol, DNA was air-dried. For PCR screening, 150 μl H₂O was added to DNA pellet. For inverse-PCR, DNA was digested in 50 μl overnight at 37° C. with 20 units of KpnI, subjected to phenol/chloroform extraction, precipitated with ethanol and re-suspended in 20 μl H2O. 5 μl DNA was self-ligated in 50 μl overnight at 16° C. with 400 units of T4 DNA ligase.

### PCR Screening

Screening was performed by PCR with Supertaq enzyme (AMBION) with 2 μl A DNA as template in 25 μl of final volume. After the first round of PCR, DNA was diluted 100 times and re-amplified with a pair of nested primers. PCR products were analyzed by electrophoresis on 1XTAE 0.8% agarose gel stained with ethidium bromide.

### Luciferase Assay

Cells in the wells of 96-well plate were lysed with 40 μl 1X luciferase lysing buffer; tissues were grinded in liquid nitrogen using Besson tissue pulverizer and extracted with 500 μl of the above buffer. Equal amounts of extracts and Steady-glow luciferase substrate were combined in the wells of 96-well plate (PROMEGA); the measurements of luciferase activity were carried out using Paradigm modular detection platform (HEWLETT-PACKARD). For in vivo analysis, luciferase activity in the tissues of wild-type mice was subtracted from the activity in the tissues of GETWISE carriers. Protein level was assessed using BCA kit and used to normalize luminescence data.

### Embryoid Bodies

Embryoid bodies (Chen, Dev Immunol, 2:29-50 (1992)) grew for 14 days in suspension. The embryoid bodies plated in 6-well plates were infected with recombinant adenovirus (2×10⁶ pfu/well) expressing either GFP or tTA (ETON BIOSCIENCES). Immediately after infection, cells were stimulated with 2 μg/ml doxycycline (MPBIO) or saline for 48 h.

### RT-PCR

RT-PCR was performed as described (Poirier, et al., Genetics, 162:831-840 (2002)). As RT-PCR control, the house-keeping gene Hprt was used and ubiquitously expressed Capn1 gene (Poirier, et al., Mamm Genome, 9:388-389 (1998)).

### Real-Time PCR

To assess Ppp1r16b/Timap mRNA level in mice, the comparative CT method was used. Experiments were conducted on Step-One Real-Time PCR system from Applied Biosystems (Foster City, Calif.). Amplification was monitored by incorporation of SYBR Green dye (Applied Biosystems) into the double strand cDNA. Hprt or Capn1 (Poirier, et al., Mamm Genome, 9:388-389 (1998)) were used as control for amplification of genomic DNA; the RQ values were reported.

### Mice

Mouse breeding and experimental procedures were approved by IACUC. Chimeric mice were generated at the University of Michigan Transgenic Animal Model Core. Transgenic mice expressing rtTA from Tek promoter (Teng, et al., Physiol Genomics, 11:99-107 (2002)), Tg(Tek-rtTA,TRE-lacZ)1425Tpr, were purchased from the Jackson Laboratory. Timap knockout mice (Heinzel, et al., Eur J Immunol,37:2562-2571 (2007)) were obtained from Professor Thomas Boehm from Max Plank Institute (Freiburg, Germany).

In vivo Expression of Timap from TIP

6-week old mice were stimulated twice daily with intra-peritoneal injection of 150 μl doxycycline (12 mg/ml) for 4 days. On the fifth day, they were stimulated once with 150 μl doxycycline (12 mg/ml) through tail vein injection. The mice were terminated 6 hours after last injection, and their lungs were collected.

### Example 1

### Targeting Ppp1r16b (Timap) Gene

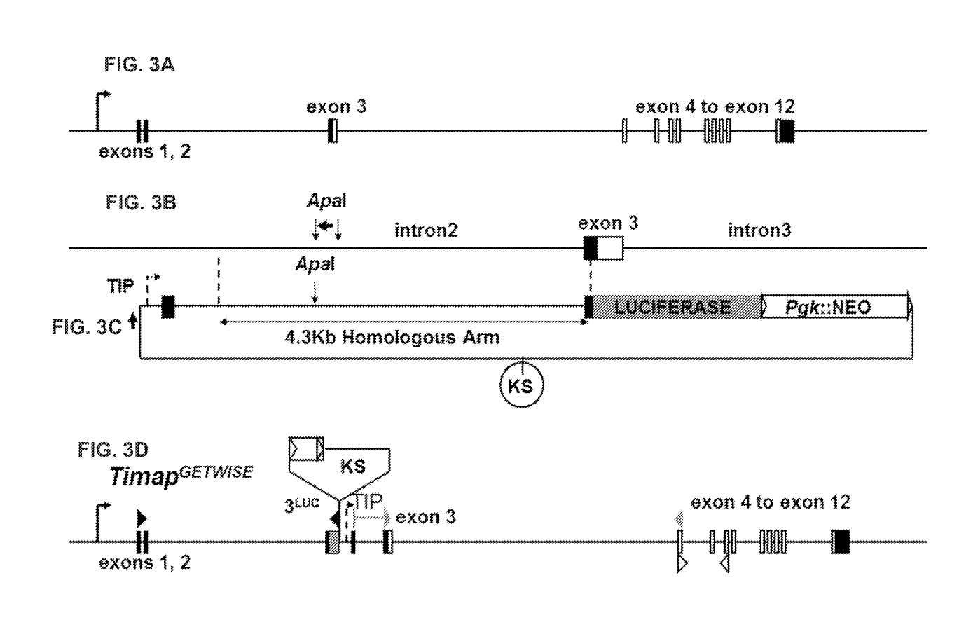

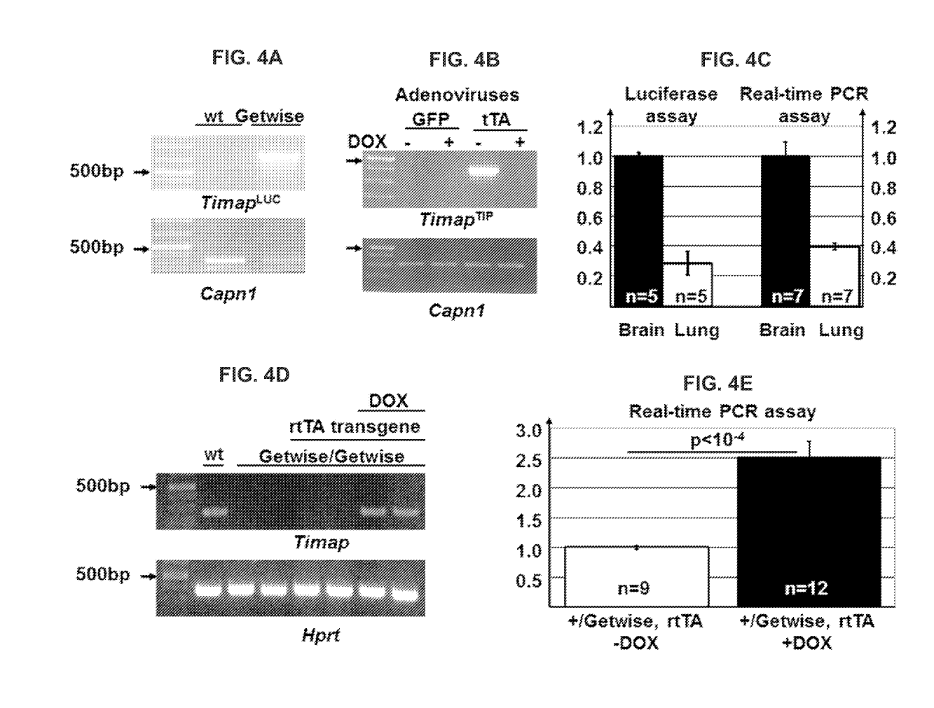

This gene encodes a novel regulatory sub-unit of protein phosphatase 1. In mouse, targeted deletion of Timap was not associated with gross anatomical abnormalities (Heinzel, et al., Eur J Immunol,37:2562-2571 (2007)); however, in vitro and in vivo data showed that TIMAP is involved in the regulation of endothelial barrier function (Csortos, et al., Am J Physiol Lung Cell Mol Physiol, 295:L440-450 (2008); Poirier, et al., Respir Physiol Neurobiol, 179:334-337 (2011)). Therefore, the ability to manipulate TIMAP expression in endothelium provides valuable information about the role of TIMAP in vascular barrier regulation. Mouse Timap (FIG. 3A) is an example of a gene a) with no coding sequence upstream the homologous region; b) containing sites for gap creation. To create homologous region, a single 4,518 by genomic fragment, consisting of 4,428 by of intron #2 and 90 by of 5′utr from exon #3 (FIG. 3B) was amplified. Deletion of an internal 206 by ApaI fragment was carried out to create the targeting gapped vector (FIG. 3C). After gene targeting in ES cells, 5 homologous recombinant clones were obtained out of 96 G418-resistant ES clones. Since Timap expression was not detected in ES cells, embryoid bodies were used for RT-PCR analysis. On the mutant allele, Timap promoter is supposed to drive the expression of luciferase reporter gene, while TIP is supposed to control the expression of Timap (FIG. 3D). It is confirmed that luciferase was expressed from Timap promoter (FIG. 4A), and TIP-driven Timap transcription was detected only under the appropriate stimulation (FIG. 4B). There was no transcriptional activity from TIP in the absence of tTA; in the presence of tTA, expression of Timap was detected only in the absence of doxycycline (Gossen, et al., Science 268:1766-1769 (1995)). After validation of GETWISE principle in vitro, the corresponding transgenic mouse line was generated. First, the ability of Timap endogenous promoter to drive the luciferase expression in mouse (FIG. 4C) was tested. Comparison of the luciferase activity in two tissues known to express Timap (brain and lung) to the Timap mRNA level showed strong correlation between the two assays. These data demonstrated that the luciferase activity in TimapGETWISE mice is indeed controlled by the endogenous Timap promoter, and allowed a researcher to employ luciferase assay to monitor Timap promoter activity in the lungs of mice subjected to LPS-induced acute lung injury (Poirier, et al., Respir Physiol Neurobiol, 179:334-337 (2011)). Next, the ability of TIP promoter to drive a tissue-specific expression of Timap conditionally was checked. By breeding, offspring homozygous for GETWISE allele (TimapGETWISE/GETWISE) and carrying Tek::rtTA transgene were produced; this transgene drives an expression of rtTA in vascular endothelium (Teng, et al., Physiol Genomics, 11:99-107 (2002)).

Analysis of mRNA from a highly vascularized organ, lung, failed to reveal Timap expression in homozygous GETWISE mice without rtTA transgene, or with rtTA transgene but without doxycycline stimulation (FIG. 4D). As expected, Timap expression was detected in lungs of homozygous GETWISE mice carrying rtTA transgene and subjected to doxycycline stimulation (FIG. 4D). These data showed that in vivo TimapGETWISE allele causes a loss of Timap expression which can be reversed in the tissue of choice upon appropriate stimulation. To prove that the method herein allows for a modulation of gene expression at the levels comparable with naturally expressed, the level of Timap mRNA in the lungs of heterozygous GETWISE mice carrying the rtTA transgene was measured. These mice carry one wild-type Timap allele and one doxycycline-inducible TimapGETWISE allele. To ensure that all the mice in study receive the exact same dose of doxycycline, the drug was delivered by injection. Alternatively, doxycycline can be administered via drinking water or food. Real-Time PCR revealed that 5 daily doses of 90 mg/kg doxycycline led to a 2.5-fold increase in Timap expression (FIG. 4E). These data demonstrated that doxycycline-inducible TimapGETWISE allele provided the level of Timap expression comparable to the expression from the wild-type allele (1.5 fold more).

### Example 2

### Targeting Smpd3 Gene

This gene encodes neutral sphingomyelinase 2 (nSMase2), enzyme converting sphingomyelin to ceramide. It has been shown that a deletion in mouse Smpd3 gene resulted in skeletal dysplasia (Aubin, et al., Nat Genet, 37:803-805 (2005); Khavandgar, et al., J Cell Biol, 194:277-289 (2011)). On the other hand, data of literature showed that over-expression of nSMase2 contributes to the pathogenesis of emphysema (Filosto, et al., Am J Respir Cell Mol Biol, 44:350-360 (2011)). Therefore, the ability to manipulate nSMase2 expression in skeletal cell lineages and pulmonary epithelium provides valuable information about the role of nSMase2 in skeletal development and lung function. Mouse Smpd3 (FIG. 5A) is an example of a gene a) with no coding sequence upstream the homologous region; b) no possibility for gap creation.

To create the homologous region, a single 4,108 bp genomic fragment, consisting of 4,018 bp of intron #2 and 90 bp of 5′utr from exon #3 was amplified (FIGS. 5B-C). After gene targeting in ES cells, 4 homologous recombinant clones out of 96 G418-resistant ES clones were obtained. Since the expression of Smpd3 in ES cells could not be detected, embryoid bodies were used for RT-PCR analysis. It was confirmed that luciferase was expressed from Smpd3 promoter, and TIP-driven Smpd3 transcription was detected only under the appropriate stimulation (FIGS. 5D-F).

### Example 3

### Targeting Ppp1r12a (Mypt1) Gene

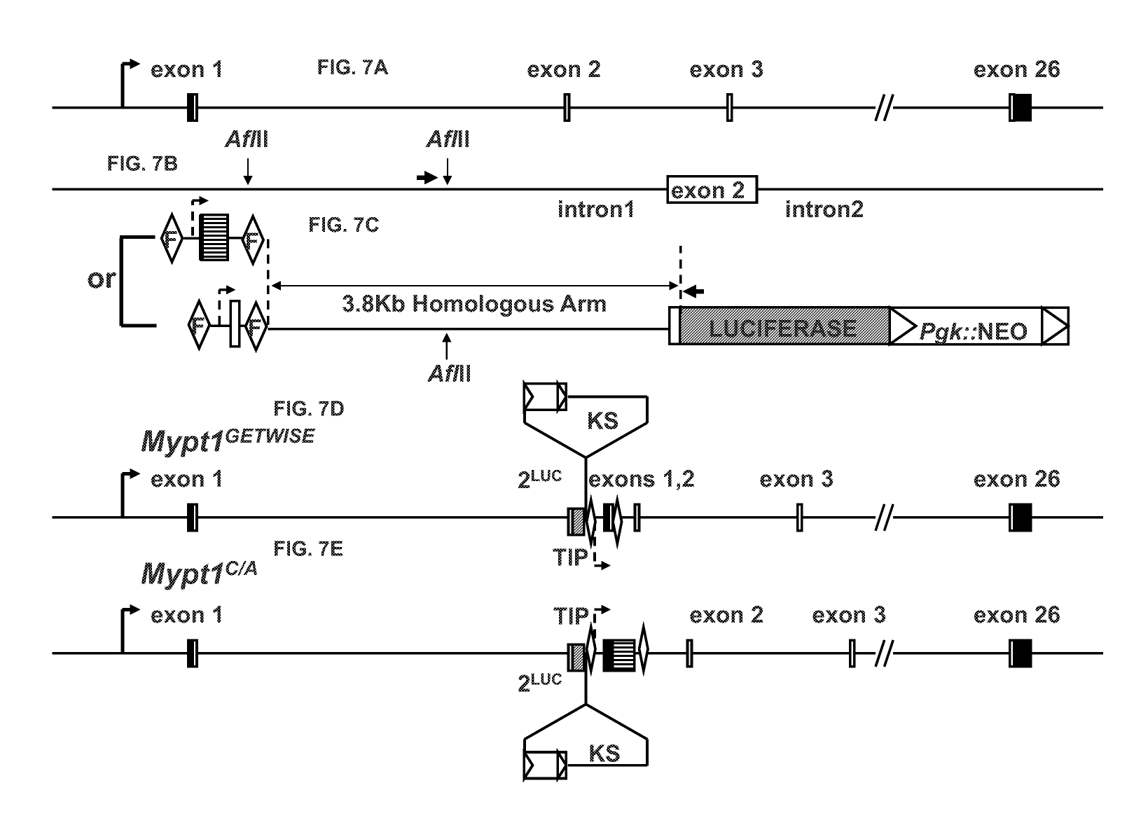

This gene encodes another regulatory sub-unit of protein phosphatase 1. Targeted deletion of mouse Mypt1 gene resulted in early embryonic lethality of homozygous mutant embryos (Okamoto, et al., Transgenic Res, 14:337-340 (2005)). In vitro data show that MYPT1 plays critical roles in vital functions such as cell division (Totsukawa, et al., J Cell Biol, 144:735-744 (1999)) and cell migration (Xia, et al., Exp Cell Res, 304:506-517 (2005)). Therefore, the ability to silence MYPT1 expression later in the development, as well as the ability to manipulate MYPT1 expression in the tissue of choice provides valuable information about the role of MYPT1 in development, physiology, and pathology. Mouse Ppp1r12a is an example of a gene a) with coding sequence upstream the homologous region; b) containing sites for gap creation (FIGS. 7A and 7B). Several MYPT1 isoforms are generated by alternative splicing (Dirksen et al. 2003; Dirksen et al. 2000). Human constitutively active mutant MYPT1 C/A cDNA (Eto, et al., Cell Motil Cytoskeleton, 62:100-109 (2005)) was inserted at PacI site of pGETWISE2.1 to create mutant GETWISE allele. Alternatively, coding sequence from exon #1 and 11 bp of adjacent intronic sequence were inserted at PacI of pGETWISE2.1 to create “classic” GETWISE allele (FIG. 7C).

To create the homologous region, a single 5,756 by genomic fragment, containing 5,643 by of intron #1 and 113 by of the coding sequence from exon #2 was amplified and cloned into pCR8/GW. The homologous region was then transferred to the GETWISE plasmids. The reverse primer from exon #2 was chosen to maintain luciferase in frame with exon #2 coding sequence. After deleting 1,911 by AflII fragment, the targeting vector containing 3,845 by of homologous region was created (FIG. 7C). Upon homologous recombination, Mypt1 promoter controls expression of a fusion protein with 147 amino acids (aa) from MYPT1 linked to luciferase; TIP controls the expression of either wild type MYPT1 splice variants (“classic” GETWISE allele, FIG. 7D) or constitutively active MYPT1C/A (mutant GETWISE allele, FIG. 7E).

### Example 4

### Targeting Sgms1 Gene

Sgms1 gene encodes sphingomyelin synthase 1 (SMS1), an enzyme generating sphingomyelin from ceramide. Sgms1 knockouts have been described recently (Yano, et al., J Biol Chem, 286:3992-4002 (2011)); they display moderate neonatal lethality, and metabolic abnormalities associated with severe deficiency of insulin secretion. Therefore, the ability to conditionally manipulate Sgms1 expression in pancreatic beta-cells provides valuable information about the role of SMS1 in diabetes.

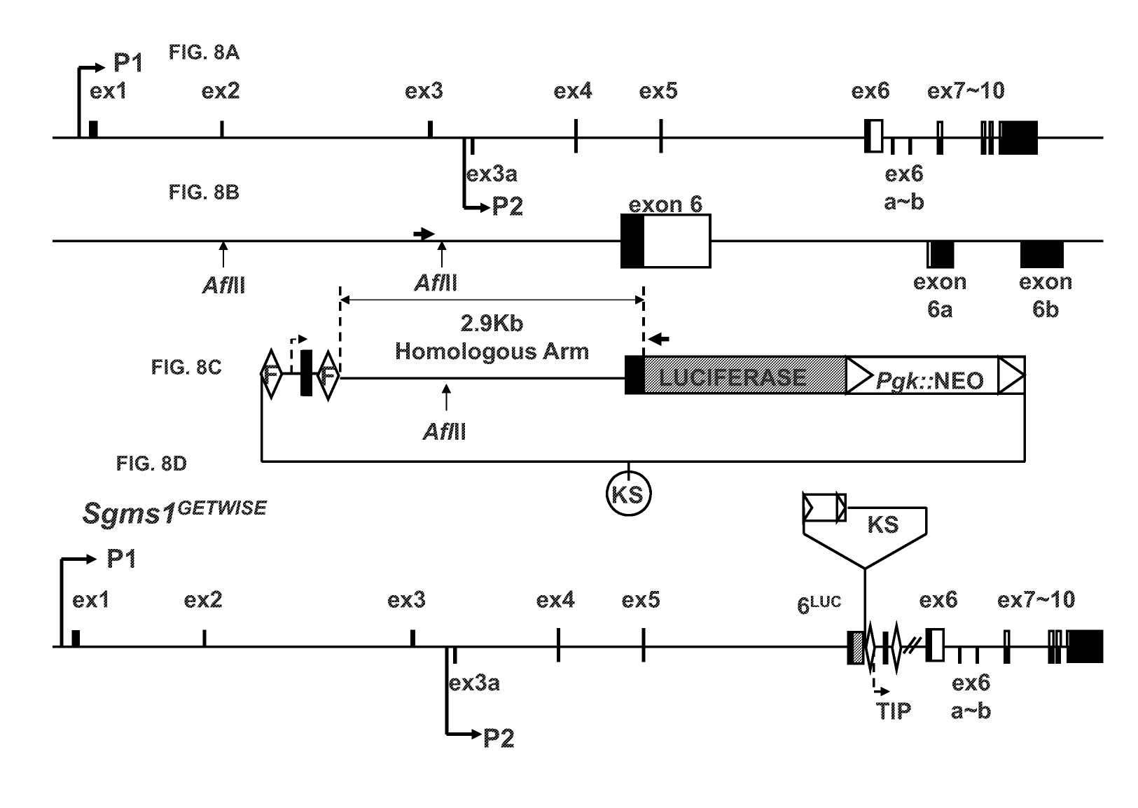

Mouse Sgms1 gene is an example of a gene a) with no coding sequence upstream the homologous region; b) containing sites for gap creation. It has at least 2 major promoters, P1 and P2 (FIG. 8A); two other promoters have been reported (Yang, et al., Gene, 363:123-132 (2005)). From all these promoters several mRNA are transcribed with exon #6 being always the first coding exon. To create homologous region, a 4,979 by genomic fragment containing 4,836 by of intron #5 and 143 by of 5′utr from exon #6 was amplified (FIG. 8B). This fragment was transferred into pGETWISE1.1; after deleting an internal 2,094 by AflII fragment, the targeting vector with a 2,885 by homologous region was created (FIG. 8C). Upon homologous recombination, Sgms1 promoters controls the expression of luciferase; TIP controls the expression of all the SMS1 isoforms (Yang, et al., Gene, 363:123-132 (2005)) (FIG. 8D).

### Example 5

### Targeting Ncrna00085 (Bachelor) Gene

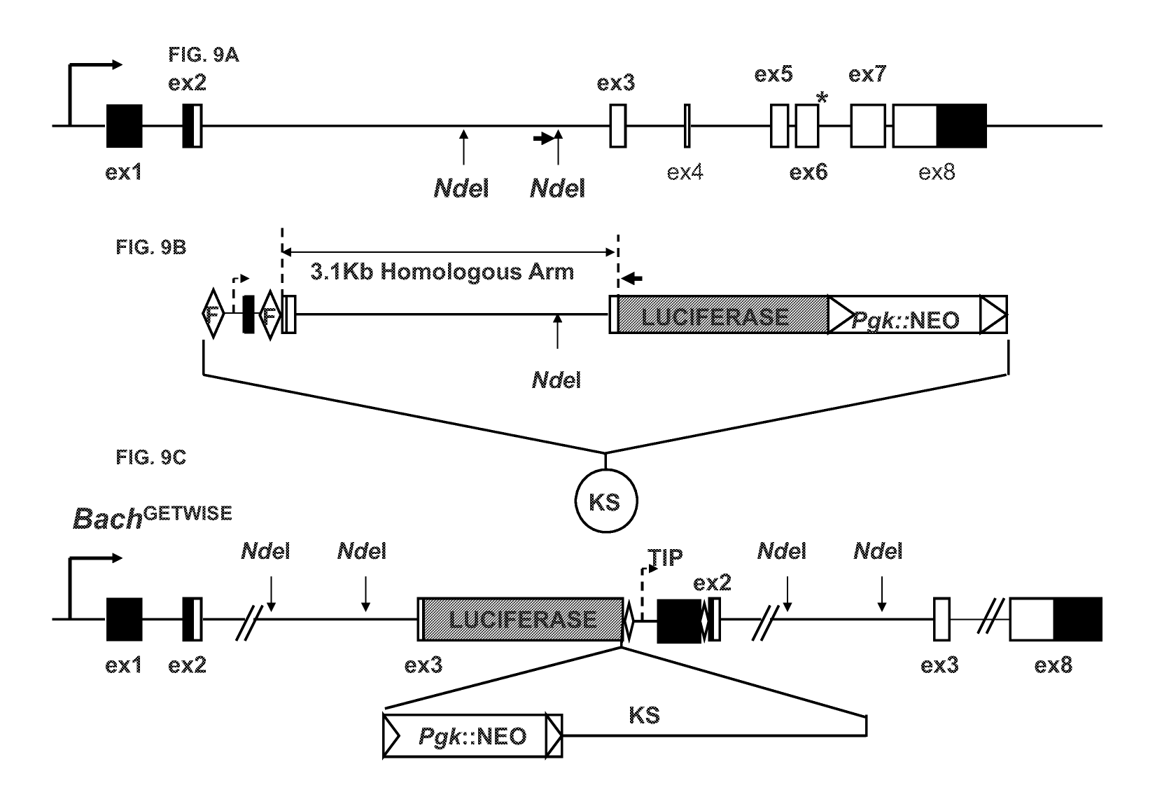

Mouse Ncrrna00085 gene was originally thought to be a non-coding RNA because of the short-size of its open reading frame. It has been found that Ncrrna00085 is a candidate gene for the mouse recessive mutation Bachelor which was identified in a screen for male sterile mutations (CP in preparation). Therefore, the ability to manipulate Bachelor expression helps a researcher determine the role of this gene in reproduction. Mouse Ncrrna00085 is an example of the gene a) with no coding sequence upstream the homologous region; b) containing sites for gap creation. It has been found two splice variants originated from Ncrrna00085 (CP in preparation) (FIG. 9A). To create homologous region, a single 4,071 by genomic fragment containing 77 by of exon #2 (including its entire coding sequence), 3,945 by of intron #2, and 49 by of exon #3 was amplified. This fragment was transferred into pGETWISE2.1 putting the luciferase in frame with exon #3 coding sequence. After deleting an internal 975 by NdeI fragment, the targeting vector with a 3,096 by homologous region was created (FIG. 9B). Upon homologous recombination, Bachelor promoter controls the expression of a fusion protein with 81aa from BACHELOR linked to luciferase; TIP controls the expression of Bachelor (FIG. 9C).

### Example 6

### Genotyping of TimapGETWISE Mice

Three approaches to genotyping of TimapGETWISE mice are described (FIGS. 12A-D). To use marker-assisted genotyping, it was necessary to identify DNA polymorphism within or nearby Timap locus. To save time, we introduced the DNA polymorphism by breeding TimapGETWISE mice with Timap knockout (KO) mice (Heinzel, et al., Eur J Immunol, 37:2562-2571 (2007)). Unlike TimapGETWISE allele, Timap KO allele carries neomycin cassette instead of exon 4 (FIG. 12A). TimapGETWISE carriers were bred with homozygous Timap knockout (KO) mice. By PCR with GETWISE primers, compound heterozygous mice were identified. These compound heterozygous mice were bred together to generate the 3 genotypes (GETWISE/GETWISE, KO/GETWISE, and KO/KO). To identify 3 genotypes, primers capable of distinguishing between TimapGETWISE and Timap KO allele were used. Both marker-assisted genotyping (FIG. 12B) and Real-Time PCR with GETWISE primers (FIG. 12C) rendered reliable results. However, the difference in the level of luciferase activity was not statistically significant within the small group of animals (FIG. 12D); therefore, the use of the luciferase activity cannot be recommended as a primary method to determine the genotype of mice.

Instead of methods mentioned above, genotyping of GETWISE carriers can be achieved by the removal of NEO cassette through breeding with CRE-expressing mice, and subsequent generation of GETWISE sub-alleles (FIGS. 14A-C). In the offspring of the GETWISE parents with and without NEO cassette, GETWISE/GETWISE mice can be easily identified by PCR. In these NEO+/−mice, the targeted gene cannot be expressed anymore from the endogenous promoters, making them knockout mice.

We claim:

1. A nucleic acid vector comprising:
a) a homologous region having a 5′ end and a 3′ end, wherein the 3′ end is homologous to an exon within a target genomic gene;
b) at least one endonuclease restriction site within the homologous region positioned to promote in-frame homologous recombination of the vector with the target genomic gene;
c) an exogenous, inducible promoter upstream of the homologous region;
d) a reporter gene immediately contiguous with the 3′end of the homologous region; and
e) a selection marker gene downstream of the reporter gene;
wherein expression of the reporter gene is controlled by a promoter of the genomic gene and expression of the target gene is controlled by the inducible promoter when the vector homologously recombines with the target genomic gene.

2. The vector of claim 1, further comprising a coding region between the inducible promoter and the homologous region, wherein the coding region comprises exons of the target genomic gene upstream of where the vector recombines with the target genomic gene.

3. The vector of claim 1, wherein the homologous region comprises 2 restriction sites for the same restriction endonuclease that are at least 100 bp apart.

4. The vector of claim 1, wherein the length of the homologous region is less than 6 kb.

5. The vector of claim 1, wherein the inducible promoter is a Tetracycline inducible promoter.

6. The vector of claim 1, wherein the selection mark gene is flanked by loxP sites.

7. The vector of claim 2, wherein the coding region comprises a splice donor site.

8. A method for producing a recombinant embryonic stem (ES) cell, comprising the steps of:
a) introducing a linearized vector of claim 1 into an ES cell,
b) selecting the homologously-recombined ES cell expressing the reporter gene.

9. The method of claim 8, where the reporter gene is luciferase.

10. The method of claim 8, where the inducible promoter is a Tetracycline inducible promoter.

11. The method of claim 9, further comprising the step of detecting the ES cells using a luciferase assay.

12. The method of claim 10 where the ES cell is from a mouse.

13. A method of creating a transgenic mouse, comprising the steps of:
a) introducing the nucleus of the ES cell of claim 12 into an anucleated mouse oocyte to produce a nuclear-transfusion mouse embryo;
b) implanting the embryo into a female mouse;
c) collecting the transgenic mouse after birth.

14. A non-human stem cell comprising the vector of claim 1.

15. The stem cell of claim 14, wherein the stem cell is an embryonic stem cell.

16. The stem cell of claim 14, wherein the stem cell is an induced pluripotent stem cell.

17. The stem cell of claim 14, wherein the stem cell is a mouse stem cell.

18. The stem cell of claim 14, wherein the stem cell is a non-human primate stem cell.

19. The stem cell of claim 14, wherein the stem cell is an adult stem cell.
